(19) United States
(12) Patent Application Publication
(10) Pub. No.: US 2026/0128119 A1
(43) Pub. Date: May 7, 2026

(54) SCREENING METHODS FOR ACINETOBACTER BAUMANNII SPOT ENZYME MODULATORS

(71) Applicant: UNIVERSITÉ LIBRE DE BRUXELLES, Bruxelles (BE)

(72) Inventors: Abel GARCIA PINO, Bruxelles (BE); Cédric Pierre GOVAERTS, Uccle (BE); Hanna AINELO, Tartu (EE); Hedvig TAMMAN, Tartu (EE); Vasili HAURYLIUK, Lund (SE)

(73) Assignee: UNIVERSITÉ LIBRE DE BRUXELLES, Bruxelles (BE)

(21) Appl. No.: 19/117,462

(22) PCT Filed: Oct. 20, 2023
(86) PCT No.: PCT/EP2023/079344

§ 371 Date: Apr. 1, 2025

(30) Foreign Application Priority Data
Oct. 20, 2022 (EP) 22202866.4

(51) Int. Cl. G16B 15/30; C12N 9/12; C12Q 1/48; G16B 15/20

(52) U.S. Cl. CPC G16B 15/30; C12N 9/1235; C12Q 1/48; G16B 15/20; C12Y 207/06005; G01N 2333/9124

(57) ABSTRACT

The present invention concerns screening methods to identify compounds that regulate the activity of Acinetobacter baumannii SpoT enzyme, in particular compounds that are able to partially or completely inhibit the hydrolase activity of said enzyme. The screening methods rely on assessing the degree of fit of candidate compounds with the three-dimensional structure of the A. baumannii SpoT protein and/or A. baumannii SpoT-ppGpp complex represented by a well-defined set of atomic coordinates. The screening methods may further rely on assessing interaction of the candidate compound with one or more amino acid residues of a region on the surface of the SpoT protein.

### FIELD OF THE INVENTION

The invention relates to the elucidation of the Acinetobacter Baumannii SpoT enzyme crystal structure, and screening methods to identify Acinetobacter Baumannii SpoT enzyme binding into the catalytic site of said crystal structure. The invention is of particular interest to the field of molecular biology, more particular in the development of drugs against antibiotic resistant Acinetobacter Baumannii.

### BACKGROUND OF THE INVENTION

The overuse and misuse of antibiotics combined with a lack of progress in the development of new antibacterial drugs have led to the emergence of pathogenic antibiotic resistant bacteria. The incidence of these bacteria (also known as “superbugs”) is increasing at an alarming rate, and thus bacterial infections are resurging as a prominent threat to human health (Ventola, The antibiotic resistance crisis, Pharmacy and therapeutics, 2015). In the last years, multiple health instances have repeatedly warned about these pathogenic antibiotic (multi)resistant bacteria and the threats they pose to human health (Michael et al., Frontiers in public health, 2013). Six of the most highly virulent and antibiotic resistant bacterial pathogens that can evade or escape commonly used antibiotics due to their increasing multi-drug resistance have been referred to recently as by the acronym “ESKAPE” (group), which consists of Enterococcus faecium, Staphylococcus aureus, Klebsiella pneumoniae, Acinetobacter baumannii, Pseudomonas aeruginosa, and Enterobacter spp.

One mechanism that these bacteria use to survive in presence of antibiotics is by the phenomenon of bacterial tolerance and persistence. Whereas the majority of a bacterial population will proliferate quickly in an infected host organism, a smaller fraction of this population will actively suppress growth. Since the majority of all clinically used antibiotics target rapidly dividing bacteria, the small population of bacteria in the persistence state will not be affected by these drugs and are able to switch back to their normal, non-persistent state post-antibiotic treatment(s). A crucial mediator to obtain the typical phenotype of a tolerant cell (also known in the art as the stringent response) is the alarmone guanosine polyphosphate (guanosine 3′, 5′-bisdiphosphate and guanosine 5′-triphosphate-3′-diphosphate), abbreviated as (p)ppGpp. The levels of (p)ppGpp are tightly regulated by the concerted opposing activities of RelA/SpoT homologue (RSH) enzymes that can both transfer a pyrophosphate group of ATP to the 3′ position of GDP (or GTP) or remove the 3′ pyrophosphate moiety from (p)ppGpp (Geiger et al., Infection and immunity, 2010).

The RelA-SpoT pair is a product of gene duplication of an ancestral factor—the ribosome-associated bifunctional RSH Rel—and the pair is limited in its taxonomic distribution to Beta- and Gammaproteobacteria (Atkinson et al., PLoS One, 2011; Mittenhuber et al., J Mol Microbiol Biotechnol, 2001).

Subfunctionalization—the partitioning of functions between two paralogues that arose through gene duplication⇒appears to have happened at least twice in Gammaproteobacteria. First, relatively soon after the duplication that gave rise to RelA and SpoT, RelA lost its capacity for alarmone hydrolysis, evolving into a monofunctional, synthetase-only (SYNTH-only) RSH. Secondly, as evidenced by a lack of sequence conservation in sites that are critical for nucleotide pyrophosphorylation, during the evolution of the Moraxellaceae lineage of Protobacteria, SpoT has likely lost its synthetase function (Atkinson et al., PLoS One, 2011). This resulted in further specialization into mono-functional (p)ppGpp hydrolase, SpoT[Hs](The uppercase “H” stands for hydrolase competent, while the lowercase “s” indicates “synthetase-incompetent”), as opposed to the bifunctional HD- and SYNTH-competent SpoT[HS] found in other Beta- and Gammaproteobacteria.

Notably, recent studies directed to A. baumannii indicated a lack of (p)ppGpp in the ΔrelA strain (i.e. an A. baumannii strain wherein the relA gene is inactivated or deleted), both with and without acute amino acid starvation induced by serine hydroxamate (SHX) (Jung et al., J Antimicrob Chemother, 2020; Perez-Varela et al., J Bacteriol, 2020). These observations are consistent with the hypothesis that RelA is, indeed, the sole source of the alarmone in this bacterium. Furthermore, consistent with the key role of (p)ppGpp-mediated signaling in bacterial virulence and antibiotic tolerance (Kundra et al., Front Microbiol, 2020), the likely ppGpp⁹ A. baumannii ΔrelA strain displays increased sensitivity to multiple antibiotics (Jung et al., J Antimicrob Chemother, 2020; Perez-Varela et al., J Bacteriol, 2020), decreased virulence in a Galleria mellonella wax moth model and deficiency in switching from the virulent opaque colony variant to the avirulent translucent colony variant (Perez-Varela et al., J Bacteriol, 2020).

Rel, RelA and SpoT all share the same conserved domain composition, indicative of a common architecture of the underlying intra-molecular allosteric regulation in long RSHs (Atkinson et al., PLoS One, 2011). When recruited to starved ribosomes, both Rel and RelA adopt a highly extended elongated conformation. In these complexes the regulatory C-terminal domain region (CTD: TGS, HEL, ZFD and RRM domains) is highly structured, while the N-terminal catalytic region (NTD: HD and SYNTH domains) and the interdomain linker regions are highly dynamic and unresolved in some structures (Arenz et al., Nucleic Acids Res, 2016; Brown et al., Nature 2016; Loveland et al., Elife 2016; Pausch et al., Cell Rep, 2020). Off the ribosome, the structural understanding of long RSHs relies on the structures of isolated NTDs of several Rel representatives (Pausch et al., Cell Rep, 2020; Hogg et al., Cell, 2004; Tamman et al., Nat Chem Biol, 2020; Mojr et al., ACS Chem Biol, 2021). While the physiological role of SpoT as a key virulence and stress tolerance factor is well established (Fitzsimmons et al., mBio, 2020; Vogt et al., Infect Immun, 2011), structural insights into SpoT are lacking.

Being one of the bacterial pathogens listed in the ESKAPE group, there is an urgent need for innovative strategies that allow effective treatment of A. baumannii bacteria. More particularly, approaches which allow screening for compounds that are able to modulate the activity of the A. baumannii SpoT enzyme would entail great value to generate novel antimicrobials.

### SUMMARY OF THE INVENTION

The present inventors have determined the full-length structure of Acinetobacter baumannii SpoT enzyme, and more particularly the complete structure of the A. baumannii SpoT-ppGpp complex in a bound active state. Obtaining this full length structure of SpoT is essential for understanding and modulating the stringent response of A. baumannii. The present findings provide key structural insights into the A. baumannii SpoT enzyme structure which enables interpreting the physiological and microbiological functions on a molecular level. The structural and biochemical data presented herein provide the long-missing structural insight into the molecular mechanism of SpoT. The inventors show that A. baumannii SpoT (SpoT_{Ab}) is a monofunctional (p)ppGpp hydrolase and uncover how its CTD is an allosteric activator of the HD hydrolase function. The structures of the full-length HD-active SpoT_{Ab} complexed with the ppGpp substrate reveal a compact monomeric conformation in which all the regulatory domains wrap around a Core subdomain that connects the pseudo-SYNTH and TGS domains. The Core is one of the intrinsically disordered regions (IDR) present in Rel and RelA when in the active synthetase state. In SpoT_{Ab}, Core and TGS cooperate to align and activate the hydrolase domain active site while translating allosteric feedback from the other regulatory domains to modulate the HD output. The inventors propose a unifying conceptual framework that rationalises the relative balance between HD vs SYNTH activities of long RSHs Rel, RelA and SpoT, fine-tuned through the entropic force produced by intrinsically disordered regions that function as conformational gatekeepers of the enzyme. By means of example and not limitation, compounds that inhibit A. baumannii SpoT hydrolase activity or even reduce enzyme A. baumannii SpoT hydrolase activity will result in a build-up of the toxic ppGpp alarmone, resulting in A. baumannii cell death.

The invention therefore relates to the following aspects:

Aspect 1. A method for identifying compounds that modulate A. baumannii SpoT activity comprising the step of employing a three dimensional structure represented by a set of atomic coordinates presented in Table 1 or a subset thereof, or atomic coordinates which deviate from those in Table 1 or a subset thereof by a root mean square deviation (RMSD) of residue over protein backbone atoms by no more than 3 Å and assessing the degree of fit of a candidate compound to said three-dimensional protein structure of A. baumannii SpoT.
Aspect 2. The method according to aspect 1, wherein the method is a method for identifying compounds that modulate A. baumannii SpoT hydrolase activity.
Aspect 3. The method according to aspect 1 or 2, wherein interactions of said candidate compound with one or more amino acid residues of a region on the surface of the protein defined by amino acid residues: Arg45, Lys46, Ser47, Tyr51, His54, His78, Asp79, Ser113, Lys140, Asp143, Asn147, Thr150, Ala153, and Lys158 of the SpoT amino acid sequence as defined by an amino acid sequence with at least 70% sequence identity to the amino acid sequence of SEQ ID NO: 1 indicate the candidate compound is a modulator of SpoT hydrolase activity.
Aspect 4. The method according to aspect 1 or 2, wherein the amino acid sequence has at least 80%, preferably at least 85%, more preferably at least 90%, even more preferably at least 95% sequence identity to the amino acid sequence of SEQ ID NO: 1.
Aspect 5. The method according to any one of the preceding aspects, wherein the amino acid sequence comprises, consists essentially of, or consists of the amino acid sequence of SEQ ID NO: 1.
Aspect 6. The method according to any one of the preceding aspects, further comprising determining a score of said candidate compound to modulate A. baumannii SpoT activity, preferably A. baumannii SpoT hydrolase activity, based on the number of interactions with said amino acid residues.
Aspect 7. The method according to anyone of the preceding aspects, further comprising comparing the conformational state of A. baumannii SpoT before and after said candidate compound binds to A. baumannii SpoT, wherein a change in conformational state is indicative for the candidate compound to be a bona fide modulator of A. baumannii SpoT activity, preferably wherein the conformational state of A. baumannii SpoT before candidate compound binding is the conformational state characterized by the atomic coordinates of Table 1.
Aspect 8. The method according to any one of the preceding aspects, wherein the method is a method for identifying compounds that partially or completely inhibit (i.e. decrease) A. baumannii SpoT hydrolase activity.
Aspect 9. The method according to any one of aspects 1 to 7, wherein the method is a method for identifying compounds that increase A. baumannii SpoT hydrolase activity.
Aspect 10. The method according to any one of the preceding aspects, further comprising testing of the ability of the candidate compounds for modulating A. baumannii SpoT hydrolase activity, preferably testing of the ability of the candidate compounds for inhibiting or increasing A. baumannii SpoT hydrolase activity.
Aspect 11. The method according to any one of the preceding aspects, wherein the candidate compound is a compound that interacts with A. baumannii SpoT via the interface between the Core domain and the regulatory C-terminal domain region.
Aspect 12. The method according to aspect 11, wherein the candidate compound is a compound that interacts with A. baumannii SpoT via the interface between the Core domain and a regulatory C-terminal domain selected from the group consisting of: TGS, HEL, ZFD, RRM, or any combination thereof.
Aspect 13. The method according to any of the preceding aspects, which is a computer-implemented method, said computer comprising an inputting device, a processor, a user interface, and an outputting device, wherein said method comprises the steps of:
a) generating a three-dimensional structure of the atomic coordinates of Table 1, or a subset thereof,
b) fitting the structure of step a) with the structure of a candidate compound by computational modeling;
c) selecting a candidate compound that possesses energetically favorable interactions with the structure of step a).
   Aspect 14. The method according to aspect 13, wherein said fitting comprises superimposing the structure of step a) with the structure of said candidate compound, optionally wherein said fitting comprises superimposing the structure of the atomic coordinates corresponding to bound ppGpp with the structure of said candidate compound.
   Aspect 15. The method according to aspect 13 or 14, wherein said modeling comprises docking modeling.
   Aspect 16. The method according to any one of aspects 13 to 15, wherein said candidate compound of step c) can bind to at least 1 amino acid residue of the structure of step a) without steric interference.
   Aspect 17. An in vitro method for identifying a compound which specifically modulates A. baumannii SpoT hydrolase activity, comprising the steps of:
a) providing a candidate compound;
b) providing an A. baumannii SpoT protein or SpoT-ppGpp complex;
c) contacting said candidate compound with said A. baumannii SpoT protein or SpoT-ppGpp complex;
d) determining the hydrolase activity of A. baumannii SpoT in the presence and absence of said candidate compound; and
e) identifying said candidate compound as a compound which modulates A. baumannii SpoT if a change in hydrolase activity is detected.
   Aspect 18. The method according to aspect 17, wherein said compound is inhibiting the hydrolase activity of A. baumannii SpoT; or wherein said compound is stimulating (i.e. increasing) the hydrolase activity of A. baumannii SpoT.
   Aspect 19. The method according to aspect 17 or 18, wherein said A. baumannii SpoT protein has at least 70% sequence identity to the amino acid sequence of SEQ ID NO: 1, preferably at least 80%, more preferably at least 90%, even more preferably at least 95% sequence identity to the amino acid sequence of SEQ ID NO: 1, most preferably wherein said A. baumannii SpoT protein is SEQ ID NO: 1.
   Aspect 20. The method according to any one of aspects 17 to 19, wherein the A. baumannii SpoT protein or SpoT-ppGpp complex is defined by the atomic coordinates of Table 1.
   Aspect 21. The method according to any one of aspects 17 to 20, wherein the specific modulation of the hydrolase activity of the SpoT protein occurs by direct binding of the candidate compound to said SpoT protein or SpoT-ppGpp complex.
   Aspect 22. Use of the crystal structure of the A. baumannii SpoT-ppGpp complex as defined by the atomic coordinates presented in Table 1, or a subset thereof, or atomic coordinates which deviate from those in Table 1, or a subset thereof, by RMSD over protein backbone atoms by no more than 3 Å for designing and/or identifying a compound which modulates A. baumannii SpoT hydrolase activity.
   Aspect 23. A computer system comprising:
a) a database containing information comprising the atomic coordinates, or a subset thereof as defined by Table 1, stored on a computer readable storage medium; and
b) an user interface to view the information.
   Aspect 24. Use of a computer system as defined in aspect 23 for designing and/or identifying a compound which modulates A. baumannii SpoT activity.
   Aspect 25. A crystal of A. baumannii SpoT-ppGpp complex, comprising a structure characterized by the atomic coordinates or a subset thereof as defined in Table 1.
   Aspect 26. The crystal according to aspect 25, obtained by crystallizing SEQ ID NO: 1.
   Aspect 27. The crystal according to aspect 25 or 26, obtained by crystallization of A. baumannii SpoT protein in a solution comprising 0.85 M Sodium citrate tribasic dihydrate, 0.1 M Tris pH 8.0, and 0.1 M Sodium chloride using a space group p21 21 21, and a unit cell: 128.791 133.761 211.328 90.00 90.00 90.00 and supplementing ppGpp to the solution prior to crystal harvesting, preferably prior to crystal harvesting at 50 mM.
   Aspect 28. A computer system, intended to generate three dimensional structural representations of an A. baumannii SpoT protein and/or SpoT-ppGpp complex, complexes of A. baumannii SpoT protein with binding compounds or modulators for analyzing or optimizing binding of compounds or modulators to said A. baumannii SpoT protein and/or SpoT-ppGpp complex, the system containing computer-readable data comprising one or more of:
(a) the coordinates of the A. baumannii SpoT protein structure listed in Table 1, optionally varied by a root mean square deviation of residue backbone atoms of not more than 3 Å, or selected coordinates thereof,
(b) the coordinates of the A. baumannii SpoT-ppGpp complex structure listed in Table 1, optionally varied by a root mean square deviation of residue backbone atoms of not more than 3 Å, or selected coordinates thereof,
(c) the coordinates of a candidate binding compound or modulator generated by interpreting X-ray crystallographic data, cryo-EM, or NMR data by reference to the coordinates of the A. baumannii SpoT protein structure and/or SpoT-ppGpp complex structure, listed in Table 1, optionally varied by a root mean square deviation of residue backbone atoms of not more than 3 Å, or selected coordinates thereof, and
(d) structure factor data derivable from the coordinates of (a), (b) or (c).
   Aspect 29. The computer system of aspect 28, wherein said computer system compares the atomic coordinates of (a) and (c), and wherein when a sterical conflict is detected the candidate compound or modulator is not considered a suitable A. baumannii SpoT protein modulator.
   Aspect 30. The computer system of aspect 28 or 29, wherein said computer system compares the atomic coordinates of (a) and (c), an wherein when no sterical conflict is detected the candidate compound or modulator is considered a suitable A. baumannii SpoT protein modulator.
   Aspect 31. A computer-readable storage medium, comprising a data storage material encoded with computer readable data, wherein the data comprises one or more of
(a) the coordinates of the A. baumannii SpoT-ppGpp complex, listed in Table 1, optionally varied by a root mean square deviation of residue backbone atoms of not more than 3 Å, or selected coordinates thereof,
(b) the coordinates of the A. baumannii SpoT-ppGpp complex structure listed in Table 1, optionally varied by a root mean square deviation of residue backbone atoms of not more than 3 Å, or selected coordinates thereof,
(c) the coordinates of a candidate binding compound or modulator generated by interpreting X-ray crystallographic data, cryo-EM, or NMR data by reference to the coordinates of the A. baumannii SpoT-ppGpp complex, listed in Table 1, optionally varied by a root mean square deviation of residue backbone atoms of not more than 3 Å, or selected coordinates thereof, and
(d) structure factor data derivable from the coordinates of (a), (b) or (c).
   Aspect 32. A computer-readable storage medium comprising a data storage material encoded with a first set of computer-readable data comprising a Fourier transform of at least a portion of the structural coordinates of the A. baumannii SpoT-ppGpp complex enzyme listed in Table 1, optionally varied by a root mean square deviation of residue backbone atoms of not more than 3 Å, or selected coordinates thereof, which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.
   Aspect 33. The computer system according to any one of aspects 28 to 30 or computer-readable storage medium according to any one of aspects 31 or 32, further comprising a database containing information on the three dimensional structure of candidate compounds or modulators which are small molecules.

Notably, each of the aspects and embodiments of the present invention outlined in the present disclosure envisage the use of the complete set of atomic coordinates of Table 1 as contained in the present disclosure, but equally envisage the use of a subset of the atomic coordinates of Table 1, wherein the subset of coordinates of Table 1 correspond to the isolated A. baumannii SpoT enzyme (i.e. SpoT in the active, bound conformation of Table 1 without ppGpp), one or more protein domains thereof, or a subset of atomic coordinates corresponding to the isolated ppGpp molecule (i.e. ppGpp without SpoT). Each of these subsets and methods of obtaining them starting from Table 1 are further detailed throughout the present disclosure.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIG. 1. Full-length monomeric A. baumannii SpoT adopts a compact “mushroom”-shaped HD-active τ-state. (a) Structure of “mushroom”-shaped SpoT_{Ab}-ppGpp complex in the τ-state. The domain organization, from N to C terminus: NTD domains hydrolase (HD), pseudo-synthetase (pseudo-SYNTH) and Core, and CTD domains, TGS, Helical (HEL), Zn-finger (ZFD) and RNA recognition motif (RRM). The ppGpp alarmone is labelled. (b) Cartoon representation the SpoT_{Ab}. The “stem” of the mushroom is formed by the enzymatic HD domain and the “cap” by the regulatory domains: NTD pseudo-SYNTH domain and the CTD domains. (c) Ribbon representation of the SpoT_{Ab}-ppGpp complex. The α6/α7 motif is held in the hydrolysis-compatible position by the folded Core domain and the TGS β-hairpin, with the Core domain communicating allosteric signals to HD from the regulatory domains. (d) The HD activity of SpoT_{Ab} is insensitive to the addition of E. coli 70S ribosomes, and non-specifically weakly inhibited by both aminoacylated and deacylated E. coli tRNA^{Val}. (e) Analytical size exclusion chromatography (SEC) of SpoT_{Ab} supports its monomeric nature in solution. (f) Experimental X-ray scattering (SAXS) analysis of SpoT_{Ab} at 8 mg/mL further confirms the monomeric nature of SpoT_{Ab}. The analysis of the normalised Kratky plot (insert) of the SAXS curve reveals folded globular shape of SpoT_{Ab}. (g) Ab initio envelope of SpoT_{Ab} reconstructed from the experimental SAXS data superimposed on the crystal structure. Comparison of both models shows that in solution the enzyme adopts the same conformation as observed in the crystal.

FIG. 2. A. baumannii SpoT is a Mn²⁺-dependent (p)ppGpp hydrolase. (a) Surface representation of SpoT_{Ab} in the τ-state. The active site cavity in the HD domain is boxed in dashed lines. (b) Zoom into the HD active site of the SpoT_{Ab}-ppGpp complex. The acidic half of the interface (residues K140, E82, D83, Y51 and R45) and the Mn²⁺ ion activate the water molecule for nucleophilic attack of the pyrophosphate bond pf ppGpp, while the basic half of the interface (K46, K158 and R161) stabilises the 3′ and 5′ phosphates of the alarmone substrate. (c) Ribbon representation of the active site of SpoT_{Ab} revealing the residues involved in coordination of ppGpp. (d) Structure of the Mn²⁺-free N-terminal region of SpoT_{Ab}, SpoT_{Ab}NTD. The HD domain is in purple (left portion of the structure) and the pseudo-SYNTH is in yellow (right portion of the structure). The disordered active site is labelled. (e) Superposition of the HD domain of SpoT_{Ab} complexed with ppGpp onto Mn²⁺-free SpoT_{Ab}. The key conformation differences in catalytically-crucial active site residues and the structural elements α3, α4 and α8 are highlighted as dashed arrows and shown in bold, respectively.

FIG. 3. The CTD controls the hydrolysis activity of SpoT by controlling the equilibrium between HD-active τ-state and HD-inactive relaxed conformations. (a, b) SAXS curves of L356D in the τ-state (a) or relaxed state (b). (c) Pseudo-atomic model of the relaxed state of SpoT_{Ab} calculated with Dadimodo (Evrard et al., 2011) using the experimental SAXS data from (b). (d) Comparison of the experimental SAXS data from the relaxed state of L356D (in grey) 715 with the theoretical scattering curve of the relaxed state (solid line) obtained from the Dadimodo model. (e) SAXS curve of RelA_{Ab} is consistent with the dimensions of the relaxed state. (f, g) SAXS curves of Rel_{B}s in the τ-state (f) or relaxed state (g). (h) Cartoon representation of experimentally observed conformational states as well as particle dimensions of long RSH enzymes.

FIG. 4. The Core domain of SpoT transduces the allosteric signal from the regulatory CTD and pseudo-SNTH to the enzymatic HD domain. (a, b) Cartoon representation of the interactions stabilising the α6-α7 motif of the HD active site (A). While the Core wraps around α7, the TGS β-hairpin forms a small hydrophobic patch that stabilises α6. These interactions preclude the movement of α6-α7 and maintain SpoT_{Ab} in a constitutive hydrolase-primed state. Key interface residues are shown as sticks and labelled. (b) The experimental SAXS curve of SpoT_{Ab}^{E379K/W382K} is consistent with the dimensions of the τ-state. Cartoon representation of the HD:Core:RRM signal transduction axis. (c) The architecture of the τ-state suggests that the RRM is locked in place via the Core and supporting interaction provided by pseudo-SYNTH, suggesting that additional tethering of RRM to pseudo-SYNTH could further stabilise the i-conformation. (d) SAXS curve of the SpoT_{Ab}^{I637D/R641D} variant in which substitutions I637D/R641D and I637A/R641A promote H-bonding and stabilise the α-helical structure, respectively, is consistent with the dimensions of the τ-state.

FIG. 5. The enzymatic output of sub functionalised RelA and SpoT RSH enzymes is evolutionarily tuned through constrains of the conformational landscape. (a) Control of the enzymatic output of the ancestral bifunctional Rel[HS]. Upon amino acid starvation Rel is recruited to starved ribosomal complexes. The ribosome-bound Rel assumes an extended conformation in which the auto-inhibitory effect of the CTD region on the SYNTH activity is released. The full activation of SYNTH activity is achieved upon binding of (p)ppGpp to an allosteric site within the NTD and release of the SYNTH inhibition by the HD domain. Conversely, off the ribosome the enzyme assumes the τ-state. In this conformation locking of the α6-α7 motif by the CTD organises the HD active site residues to promote the HD activity. This, in turn, strongly inhibits the SYNTH activity via inter-NTD regulation. The full activation of either SYNTH or HD requires allosteric signalling from CTD to NTD enzymatic domains. (b, c) Evolution of SpoT as a predominantly dedicated hydrolase involved the loss of the allosteric control of the NTD by (p)ppGpp as well as by the ribosome. In bifunctional SpoT[HS] present in the majority of Gamma- and Betaproteobacteria, while the equilibrium is strongly shifted towards the HD-active τ-state, the enzyme is capable of inefficient (p)ppGpp synthesis in the relaxed state (B). Sub functionalization of SpoT in Moraxellaceae has resulted in the monofunctional hydrolase SpoT[Hs], which naturally populates only the compact τ-state and is SYNTH-inactive. (d) Sub functionalization of Gamma- and Betaproteobacterial RelA[hS] constitutes the other extreme case of evolutionary restriction of the conformational dynamics of the ancestral Rel[HS]. While losing its HD activity, RelA retains all the allosteric regulatory elements of Rel. Being a dedicated (p)ppGpp synthetase enzyme, off the ribosome RelA does not assume the τ-state. Instead, it predominantly populates the functionally frustrated resting state equivalent to the relaxed state of Rel, primed to assume the elongated ribosome-associated state triggered by the 70S ribosome, uncharged tRNA and alarmones during stringency. Circles represent catalytic centres in their different activation states.

FIG. 6. Key amino residues for candidate compound screening Visualization of the binding pocket of A. baumannii SpoT enzyme bound to ppGpp. A non-limiting group of key amino acid residues which are preferred residues for a successful candidate compound (i.e. a candidate A. baumannii SpoT enzyme modulator) to bind with are annotated.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3 or more, 4 or more, 5 or more, 6 or more, or 7 or more etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Amino acids are referred to herein with their full name, their three-letter abbreviation or their one letter abbreviation.

It is evident that the terms such as “(candidate) compound”, “(candidate) binding compound”, “(candidate) ligand”, and (candidate) modulator may be used interchangeably to describe the invention.

A skilled person is aware of standard molecular biology techniques that are available in the art (Green and Sambrook, Molecular cloning: a laboratory manual 4^{th} Ed, Cold Spring Harbor laboratory press, 2012; Ausubel et al., Current protocols in molecular biology, John Wiley and Sons, 1989; Perbal, A Practical Guide to Molecular Cloning, John Wiley & Sons, 1988; Watson et al., Recombinant DNA, Scientific American Books, New York; Birren et al. Genome Analysis: A Laboratory Manual Series, Vols. 1-4 Cold Spring Harbor laboratory press, New York, 1998).

The term “RSH enzymes” as used herein is an abbreviation for the group of RelA/SpoT homolog enzymes. RSH enzymes derive their name from the sequence similarity to the RelA and SpoT enzymes of Escherichia coli. RSH enzymes comprise a family of enzymes that synthesize and/or hydrolyze the alarmone ppGpp and play a central role in the bacterial stringent response. So-called “Long” RSH enzymes that comprise a hydrolase and synthetase domain have been identified in a vast and diverse amount of bacteria and plant chloroplasts, while specific RSH enzymes that only synthesize or hydrolyze (p)ppGpp have also been discovered in disparate bacteria and animals respectively. In the art, RSH enzymes are stratified into three groups based on their activity: long RSH enzymes, small alarmone synthetases (SASs), and small alarmone hydrolases (SAHs). These initial groups have been further classified in a plethora of subgroups (Atkinson et al., Plos One, 2011). Long RSHs comprise two catalytic domains (the (p)ppGpp hydrolase (HD) domain and the (p)ppGpp synthetase (SYN) domain) and a C-terminal protein domain that is involved in regulation of the enzyme. In contrast, both SASs and SAHs lack the conserved C-terminal regulatory domain. According to the art, long RSHs are most broadly distributed and often further comprise TGS (ThrRS, GTPase, and SpoT) and ACT (Aspartokinase, Chorismate mutase and TyrA) domains in their C-terminal domain, which may play a role in sensing stress signals such as starvation signals and transducing said signal to the catalytic domain.

The term “stringent response”, used interchangeably in the art with “stringent control” is indicative for a stress response mediated by RSH enzymes in response to various stress conditions including the non-limiting examples of amino acid starvation, fatty acid limitation, iron limitation, and heat shock. In such stress conditions, the stringent response mediates a profound shift in gene expression from a program focused on growth to a gene expression profile that allows prolonged survival in a stationary phase following failure of aminoacyl-tRNA pools to support protein synthesis. Hence, the stringent response is a key mediator in the process of bacterial persister cell formation. The stringent response has been extensively described in the art (inter alia in Traxler et al., Mol Microbiol, 2013). The stringent response is governed by the alarmones guanosine 5′, 3′ bispyrophosphate and guanosine pentaphosphate (ppGpp and pppGpp respectively). (p)ppGpp accumulation will actively inhibit resource intensive cellular processes including replication, transcription and translation. (p)ppGpp has been demonstrated to bind to RNA polymerase proximal to its active site which causes a cessation of transcription of stable RNAs. Furthermore, (p)ppGpp decreases the half-life of the open complex at most promoters that have been tested in the art, hereby mediating a strong down regulation of promoters with intrinsically short half-lives, such as those of stable RNA genes. Taken together, the stringent response includes a large-scale down regulation of the translation apparatus (Barker et al., J Mol Biol, 2001). Additionally, (p)ppGpp has been shown to upregulate transcription of promoters that act on amino acid biosynthesis genes together with RNA-polymerase binding transcription factor DksA (Paul et al., PNAS USA, 2005).

“Persister cells”, or short “persisters” as used herein is used to describe a population of bacterial cells that are in or going into a metabolically inactive (i.e. dormant) or near dormant state characterized by no growth or very slow growth, also called a stationary phase (Lewis, Nature Reviews Microbiol, 2007). Typically, in an infected organism which is optionally being treated with antibiotics, persister cells amount to a small fraction of the total bacterial population present in said infected organism. Upon termination of antibiotics treatment, persister cells can leave their dormant state and return to a growth-focused gene expression signature, and expand to a full size bacterial infection. Persister cells are often described to constitute a subpopulation of bacteria that, due their slow growth rate, become highly tolerant to antibiotics. Persistent bacterial cells may arise from a genetic change and/or a metabolic change. A skilled person is aware that persistence of a bacterial cell is associated with the emergence of antibiotic resistance (Windels et al., Bacterial persistence promotes the evolution of antibiotic resistance, 2019). Links between (p)ppGpp production and formation of bacterial persister cells have been described (inter alia in Korch et al., Mol Microbiol, 2003). Persister cells may form within biofilms.

The term “biofilm” is commonly used in the art and is indicative for a collection (i.e. aggregate) of (syntrophic) microorganisms such as bacteria wherein the different cells adhere to each other, and optionally the surface contacting the cells, or a portion of the cells. Biofilms are further characterized by a viscous extracellular matrix comprising extracellular polymeric substance (EPS) produced by microorganisms of the biofilm, wherein the microorganisms are embedded by the EPS. Biofilms may be formed both in or on organisms and on non-living surfaces in a wide array of different settings. Biofilms are complex microbiological systems wherein the microorganism comprised in said biofilm may be organized into a functional unit or functional community (Lopez et al., Biofilms, Cold Spring Harbor perspectives in biology, 2010).

The term “alarmones” is known to a skilled person and refers to intracellular signal molecules that are produced as a consequence of and in response to environmental cues. The main function of alarmones is to regulate gene expression. Typically, the concentration of alarmones rises when a cell experiences stressful environmental factors. (p)ppGpp is considered a textbook example of an alarmone (Hauryliuk et al., Nat Rev Microbiol, 2015). A skilled person appreciates that the term “(p)ppGpp” encompasses both guanosine pentaphosphate (pppGpp) and guanosine tetraphosphate (ppGpp).

As indicated above, the findings of the inventors allows for the provision of a screening method to modulate A. baumannii SpoT activity. The atomic coordinates of the A. baumannii SpoT enzyme and more particularly the SpoT-ppGpp complex contained in Table 1 enable these methods.

Hence, in a first aspect the invention is directed to a method for identifying compounds that modulate A. baumannii SpoT enzyme activity comprising the step of employing a three dimensional structure represented by a set of atomic coordinates presented in Table 1 or a subset thereof, or atomic coordinates which deviate from those in Table 1 or a subset thereof by a root mean square deviation (RMSD) of residue over protein backbone atoms by no more than 3 Å and assessing the degree of fit of a candidate compound to said three-dimensional protein structure of A. baumannii SpoT and/or SpoT-ppGpp complex.

“Acinetobacter baumannii” as used herein is to be interpreted according to the commonly accepted meaning in the art, i.e. the opportunistic Gram-negative bacterial pathogen in humans (Domain: Bacteria; Phylum: Pseudomonadota; Class: Gammaproteobacteria; Order: Pseudomonadales; Family: Moraxellaceae; Genus: Acinetobacter; Species: A. baumannii). “Acinetobacter baumannii SpoT enzyme”, interchangeably used with the terms “SpoT enzyme”, “SpoT”, and SpoT_{Ab} refers to the bifunctional (p)ppGpp synthetase/guanosine-3′,5′-bis(diphosphate) 3′-pyrophosphohydrolase expressed by the bacterium Acinetobacter baumannii.

“RMSD”, “root-mean-square deviation”, or “root-mean-square deviation of atomic positions” as used herein is indicative for a quantitative measurement of similarity between two or more protein structures, more specifically the measure of the average distance between the (backbone) atoms of superimposed proteins. The RMSD value is commonly calculated by the formula:

$$R M S D = √ ( ( \frac{1}{N} ) {\sum }_{i = 1}^{N} {δ}_{i}^{2}$$

wherein δ is the distance between atom I and the mean position of the N equivalent atoms, or alternatively a reference structure. When calculating the RMSD for backbone, heavy atoms values are calculated for C, N, O, and Cα or solely for Cα. As a RMSD value represents a distance, the value is commonly expressed in the art in A (Angstrom). 1 Å corresponds to 10⁻¹⁰ m, or 0.1 nanometer. A skilled person appreciates that a lower RMSD indicates smaller structural differences between the compared structures, or between a structure and a reference structure. In certain embodiments, the atomic coordinates used in the method deviate by no more than 2.5 Å, preferably no more than 2 Å, more preferable no more than 1.5 Å, even more preferably no more than 1 Å from the atomic coordinates of Table 1.

The term “atomic coordinates” as used herein refers to a position of an atom in space, typically expressed by a set of X, Y, and Z Cartesian coordinates and the chemical element each atom represents. Atomic coordinates for a certain protein structure are typically combined in atomic coordinate data files, which can have various data formats, including the formats of Table 1 as enclosed in this specification. Other non-limiting data formats include Protein Data Bank (PDB) format or various text formats. Minor variations in the atomic coordinates are envisaged, and the claims have been formulated with the intent of encompassing such variations. In certain embodiments, the atomic coordinates further contain additional information. It is evident to a skilled person that a three-dimensional rigid body rotation or a translation of said atomic coordinates does not alter the structure of the molecule. It is evident that, since the atomic coordinates disclosed herein are a relative collection of points delineating a three-dimensional structure, a distinct set of coordinates may define a similar or identical three-dimensional structure. In view hereof, multiple computer analysis tools and programs have been developed to assess whether a molecular structure bears similarity to the structured defined by the atomic coordinates, or a subset of atomic coordinates described herein in Table 1. By means of illustration and not limitation, a suitable software application for conducting such analyses is the Molecular Similarity program of QUANTA (Molecular Simulations Inc., San Diego, CA). The Molecular Similarity program and consorts permit extensive comparison between different structures, different conformations of the same structure, and different parts of the same structure. The method of comparison typically involves a step of calculating one or more optimal translations and rotations required such that the RMSD of the fit over the specified pairs of equivalent atoms is an absolute minimum. Therefore, atomic coordinates of the A. baumannii SpoT protein or SpoT-ppGpp complex, or fragments leading to the atomic coordinates in Table 1 by translations and/or rotations are within the scope of the present invention.

The method described herein may be performed using the atomic coordinates presented in Table 1 (which in their totality represent an active state of A. baumannii SpoT enzyme bound to ppGpp, indicated throughout the present disclosure as the (A. baumannii) SpoT-ppGpp complex) or may alternatively be performed using a subset thereof (such as a subset defining the structure of the active bound A. baumannii SpoT excluding the ppGpp molecule). The size of the subset is not particularly limiting, however a skilled person appreciates that the performance of the method described herein benefits from increasing sizes of said subset derived from Table 1. By means of illustration and not limitation, the subset may comprise 20%, preferably 40%, preferably 50%, preferably 60% preferably 70%, preferably 80%, preferably 90% of the atomic coordinates presented in Table 1. The term “subset” as defined herein indicates a portion of the atomic coordinates of Table 1. By means of illustration and not limitation, a possible subset in the context of the invention is the subset of coordinates defining the isolated ppGpp molecule part of the A. baumannii SpoT-ppGpp complex (i.e. exclusively the group of atomic coordinates of Table 1 annotated as “G4P” coordinates). An alternative possible subset is the subset of coordinates defining the A. baumannii SpoT enzyme part of the A. baumannii SpoT-ppGpp complex (i.e. the atomic coordinates of Table 1 excluding those annotated as “G4P”). Hence, the method described herein may be performed on the set of atomic coordinates presented in Table 1 as a whole. Alternatively, the method described herein may be performed on the subset of atomic coordinates presented in Table 1 annotated to be “G4P” coordinates. Yet alternatively, the method described herein may be performed on the subset of atomic coordinates presented in Table 1 not annotated to be “G4P” coordinates.

Yet an alternative possible subset is the subset of coordinates defining the key amino acid residues of the A. baumanni SpoT enzyme in the SpoT-ppGpp complex, such as the key amino acids Arg45, Lys46, Ser47, Tyr51, His54, His78, Asp79, Ser113, Lys140, Asp143, Asn147, Thr150, Ala153, and Lys158.

“Degree of fit”, or alternatively “goodness of fit” in the art, is an expression to indicate the likelihood that a certain candidate binding mode represents a favourable binding interaction and allows ranking of different ligands relative to each other. In certain embodiments, the degree of fit between the three-dimensional A. baumannii SpoT structure (or the three-dimensional SpoT-ppGpp complex structure) and the candidate SpoT modulator is expressed with a numerical value. In alternative embodiments, the degree of fit is expressed by an illustration of the superimposed A. baumannii SpoT structure (or SpoT-ppGpp complex) and the compound structure. In certain embodiments, the degree of fit of a ligand is expressed relative to the fit of a known ligand of the A. baumannii SpoT protein. A degree of fit may be expressed as an absolute or relative value, depending on the methodology used for calculating the quantitative score. When the degree(s) of fit are expressed as absolute values, this absolute value corresponds to a score given to a candidate compound based on the number of interactions in silico predicted to occur with a set of atomic coordinates as described in Table 1 herein, and/or with a set of amino acid residues in said region on the surface of the protein as described herein. Said number of interactions can be one or more such as two, three, four, five, six, seven, eight, nine, ten, more than ten, or all amino acid residues in said region on the surface of the protein as defined herein. In certain embodiments, the atomic coordinates described in Table 1, and/or the amino acid residues cited herein to constitute a surface region of the protein are further abstracted to a pharmacophore, i.e. a set of molecular features required for molecular recognition of a ligand by a biological macromolecule, herein the candidate compound and the A. baumannii SpoT protein. In certain embodiments, a degree of fit (i.e. a fitting score) of 2.4 is used as threshold for candidate compounds to be considered for further examination and/or validation. In alternative embodiments, a fitting score of 3.0 is used. In alternative embodiments, a fitting score of between 2.4 and 3.0 is used, preferably between 2.5 and 3.0, between 2.7 and 3.0, between 2.9 and 3.0. In alternative embodiments a fitting score of between 2.4 and 2.9 is used, preferably between 2.4 and 2.7, between 2.4 and 2.5. In certain embodiments, a variable fitting score threshold is used depending on the molecular weight of candidate compounds. In further embodiments, candidate compounds of 301 Da to 330 Da have a fitting score threshold of 2.4, candidate compounds of 331 Da to 380 Da a fitting score threshold of 2.5, candidate compounds of 381 Da to 420 Da a fitting score threshold of 2.7, candidate compounds of 421 Da to 490 Da a fitting score threshold of 2.9, and candidate compounds of 491 Da to 540 Da a fitting score threshold of 3.0. When the degree of fit is a relative value, this degree of fit may be expressed relative to a reference compound known to modulate the activity of the A. baumannii SpoT protein. In such embodiments, a candidate compound is considered a bona fide modulator of A. baumannii SpoT when the degree of fit is at least 50%, preferably at least 60%, at least 70%, at least 80%, at least 85%, at least 90%, most preferably at least 95% to a reference compound known to modulate the activity of A. baumannii SpoT enzyme. It is evident that a direct comparison between the degrees of fit of multiple ligands may be derived from this initial score. Numerous scoring functions or mechanisms have been described in the art (inter alia in Fu and Zhang, Interdiscip Sci, 2019), and it is evident that different scoring functions are suitable for generating a degree of fit between a candidate compound and the A. baumannii SpoT protein. For example, when using the AMBER scoring function (Wang et al., J Comput Chem, 2004), a candidate compound is considered to be a candidate bona fide modulator when a docking score threshold is met. In certain embodiments a docking score threshold of −8.9 kcal/mol is used. In certain embodiments a docking score threshold of between −8.9 kcal/mol and −10.5 kcal/mol is used. In further embodiments a docking score threshold of between −9.4 kcal/mol and −10.5 kcal/mol is used. In yet further embodiments a docking score threshold of between −9.7 kcal and −10.5 kcal/mol is used. In alternative further embodiments a docking score threshold of between −8.9 kcal/mol and −10.3 kcal/mol is used. In further embodiments a docking score threshold of between −8.9 kcal/mol and −9.7 kcal/mol is used. In further embodiments a docking score threshold of between −8.9 kcal/mol and −9.4 kcal/mol is used. In alternative embodiments, a docking score threshold of −10.5 kcal/mol was used. In yet alternative embodiments, a variable docking score threshold was used, preferably based on the molecular weight of the candidate compounds. In further embodiments, compounds with a molecular weight of 301 Da to 330 Da are assigned a docking score threshold of −8.9 kcal/mol, compounds with a molecular weight of 331 Da to 380 Da are assigned a docking score of −9.4 kcal/mol, compounds with a molecular weight of 381 Da to 420 Da are assigned a docking score of −9.7 kcal/mol, compounds with a molecular weight of 421 Da to 490 Da are assigned a docking score of −10.3 kcal/mol, and compounds with a molecular weight of 491 to 540 Da are assigned a docking score threshold of −10.5 kcal/mol.

“In silico analysis” as defined herein is indicative for an analysis performed on a computing system or by use of a computer simulation system that is guided by a set of specific instructions such as a molecular docking computer program or tool. “Molecular docking” indicates a method that allows prediction of a binding and/or preferred orientation of one molecule to a second molecule when bound to each other to form a stable complex. Hence, it is understood that molecular docking software predicts the behavior of molecules in binding sites of target proteins. Molecular docking software tools and programs that allow assessing of specificity of a candidate molecule or candidate compound against a particular target have been described in the art. Molecular docking software allows searching for complementarities between shape and/or electrostatics of binding sites surfaces and ligands. A molecular docking process can be separated into two major steps: searching and scoring. Numerous examples of different docking tools and programs have been described and are thus known to a skilled person (Pagadala et al., Biophys Rev, 2017). Two main popular molecular docking approaches have been described, a first being molecular docking relying on shape complementarity or geometric matching, and a second one relying on simulating the docking process whereby ligand-protein pairwise interaction energies are calculated.

“Modulator” as used herein indicates a molecule that influences one or more (enzymatic) activities of one or more proteins upon interaction with (and/or binding of) said protein. As used herein, the modulating effect of the modulators described herein is intended to act on the hydrolase activity of the A. baumannii SpoT protein as defined herein. Hence, a modulator as discussed herein can refer to a molecule that is a hydrolase activator or hydrolase inhibitor. Preferred modulators in the context of the present invention are hydrolase inhibitors. Such modulators will disturb the balance between ppGpp formation and breakdown, causing a buildup of the toxic ppGpp alarmone in the A. baumannii bacterium, ultimately leading to cell death. The principal binding site of a modulator is commonly termed the orthosteric site, which may be for example the active site of an enzyme where it engages in a binding with (a) substrate(s). Additionally, modulators may exert their activity by binding to a second binding site, commonly referred to as an allosteric binding site. Both orthosteric and allosteric modulators, preferably hydrolase inhibitors, are envisaged in the context of the present invention. It is appreciated by a skilled person that orthosteric modulators such as orthosteric inhibitors will compete for A. baumannii SpoT binding with ppGpp. Generally envisaged allosteric modulators are described further throughout the present description.

In the context of the invention, a modulator is said to be an “inhibitor” when as a consequence of interaction between the modulator and the target protein, in the context of the present invention the A. baumannii SpoT protein, is that at least the hydrolase activity of said target protein is reduced, either partially (i.e. to a certain degree) or completely. In the latter case it is understood that due to interaction with the modulator an enzymatic activity of the target protein is diminished to 0%, or below an activity level that can be measured by methods available in the art (such as in Gratani et al., PLoS genet, 2018). “Inhibition” as used herein refers to the inhibition of a process, herein a molecular process, more particularly SpoT enzyme hydrolase activity. It is evident to a skilled person that inhibition can be used interchangeably with the term “attenuation”. In certain embodiments, the inhibitor selectively inhibits A. baumannii SpoT hydrolase activity. In alternative embodiments, the inhibitor selectively inhibits A. baumannii SpoT hydrolase activity in addition to the hydrolase and/or synthetase activity of at least one other A. baumannii enzyme. In yet alternative embodiments, the inhibitor selectively inhibits A. baumannii SpoT hydrolase activity in addition to the hydrolase and/or synthetase activity of at least one other enzyme expressed by a bacterium selected from the group consisting of: Enterococcus faecium, Staphylococcus aureus, Klebsiella pneumoniae, Acinetobacter baumannii, Pseudomonas aeruginosa, and Enterobacter spp.

Both reversible and irreversible inhibitors are envisaged herein. “Reversible inhibition” and “irreversible inhibition” are known terms to person skilled in the art and are commonly used to further specify a type of enzyme inhibitor. Binding of an inhibitor to an enzyme is either reversible or irreversible. Irreversible inhibitors usually react with the enzyme and induce a chemical change or modification (e.g. via covalent bond formation). These inhibitors typically modify key amino acid residues needed for enzymatic activity. In contrast, reversible inhibitors bind non-covalently and different types of inhibition have been described depending on whether these inhibitors bind to the enzyme, the enzyme-substrate complex, or both. Methods to measure the dissociation constant (Kd) of a reversible inhibitor are well known to a skilled person (Pollard, Mol Biol Cell, 2010).

The term “dissociation constant”, or “Kd” used herein is an equilibrium constant that quantitatively expresses the propensity of a larger object to separate or dissociate reversibly into smaller components. It is known to a person skilled in the art that the dissociation constant is routinely used to quantify the affinity between a ligand and a drug and is therefore indicative for how tightly or strongly a ligand binds to its target protein. The affinity of a ligand for a protein is associated with the amount of non-covalent intermolecular interactions between the ligand and the protein such as hydrogen bonds, electrostatic interactions, hydrophobic interactions and Van der Waals forces. In addition, the concentration of other molecules present in the proximal environment the ligand-protein interaction takes place in can also affect affinities. This observation is known to a skilled person as molecular crowding (Rivas et al., Trends Biochem Sci, 2016).

In certain embodiments wherein a three-dimensional structure is employed corresponding to a subset of atomic coordinates presented in Table 1, this subset is selected such that the retained atomic coordinates correspond to the N-terminal catalytic region (NTD) portion of A. baumannii SpoT, optionally supplemented with the atomic coordinates of the ppGpp molecule. In alternative embodiments wherein a three-dimensional structure is employed corresponding to a subset of atomic coordinates presented in Table 1, this subset is selected such that the retained atomic coordinates correspond to the C-terminal domain region (CTD) portion of A. baumannii SpoT, optionally supplemented with the atomic coordinates of the ppGpp molecule.

In embodiments wherein a three-dimensional structure is employed corresponding to a subset of atomic coordinates presented in Table 1, this subset is selected such that the retained atomic coordinates correspond to one or more A. baumannii SpoT domains selected from the group consisting of: NTD domains hydrolase (HD), pseudo-synthetase (pseudo-SYNTH), Core, TGS, helical (Hel), Zn-finger (ZFD), and RNA recognition motif (RRM). Optionally, the subset is selected such that the retained atomic coordinates define the HD domain and consequently correspond to residues 1 to 194 of SEQ ID NO: 1. Optionally, the subset is selected such that the retained atomic coordinates define the pseudo-SYNTH domain and consequently correspond to residues 195 to 332 of SEQ ID NO: 1. Optionally, the subset is selected such that the retained atomic coordinates define the Core domain and consequently correspond to residues 333 to 380 of SEQ ID NO: 1. Optionally, the subset is selected such that the retained atomic coordinates define the TGS domain and consequently correspond to residues 381 to 453 of SEQ ID NO: 1. Optionally, the subset is selected such that the retained atomic coordinates define the helical domain and consequently correspond to residues 457 to 536 of SEQ ID NO: 1. Optionally, the subset is selected such that the retained atomic coordinates define the ZFD domain and consequently correspond to residues 560 to 605 of SEQ ID NO: 1. Optionally, the subset is selected such that the retained atomic coordinates define the RRM domain and consequently correspond to residues 618 to 688 of SEQ ID NO: 1. A skilled person appreciates that each of these subsets (i.e. domains) or a group of these subsets may form the basis for a method that aims to identify allosteric modulators of A. baumannii SpoT hydrolase activity, preferably for a method that aims to identify allosteric inhibitors of A. baumanni SpoT hydrolase activity.

Optionally, the candidate compounds that are identified by the screening method subject of the invention are small molecule compounds. The term “small” as used as used herein, e.g. in terms such as “small molecule” or “small compound” or “small candidate (binding) compound” refers to a low molecular weight compound that is organic, inorganic or organometallic and has a molecular weight of less than 1000 Da, and for instance has a molecular weight of less than 900 Da, or less than 750 Da, or even less than 600 Da. Small compounds used in the methods herein may be naturally occurring or solely occurring due to chemical synthesis.

The method subject of the invention is a method for identifying compounds that modulate A. baumannii SpoT hydrolase activity. The term “hydrolase” used herein is indicative for a class of enzymes or enzyme domains that utilize water to disrupt, or break a chemical bond, generating two distinct molecules from one molecule. Hence, it is evident that hydrolase refers to an enzyme capable of conducting hydrolysis. Unless explicitly mentioned, by hydrolase activity herein is meant the hydrolysis of (p)ppGpp, i.e. removal of the 3′ pyrophosphate moiety from (p)ppGpp. Conversely, the term “synthetase” as used herein refers to an enzyme, or enzyme domain that catalyzes a synthesis process. In the context of the invention, “synthetase activity” refers to the transfer of pyrophosphate from ATP to the 3′ position of the ribose of GDP or GTP.

In certain embodiments, the amino acid sequence of A. baumannii SpoT enzyme as used by the (screening) methods described herein has at least 70% sequence identity, preferably at least 75% sequence identity, more preferably at least 80% sequence identity, more preferably at least 85% sequence identity, more preferably at least 90% sequence identity, even more preferably at least 95% sequence identity to the amino acid sequence of the Acinetobacter baumannii SpoT as defined in SEQ ID NO: 1:

MPGEEVSQAKQQLKLIIDPYLSVSEVEKVLAACDFGDLAHTGITRKSGEP
YILHPIAVSCILANMRLDPETLMAALLHDVIEDTQYTKDDIIERFGQTVA
ELVDGVTKLSQSSDKEYNKAASFRKILQATLQDPRVIIIKLADRYHNMTT
LGALRPDKRARIAQETFDIFVPMARLVGMNEMADNLENLCYQNLDLDMFD
NVQNALLQTKPERCKYQSIWEQNLAELLHNYHIQGRIKKKNNNIELLRHF
VKNEMDLQELTHSHAFEIVLQSIADCDRLVAALKENFQVIQYQDHIRRPL
PGGNQSLMIKLKGEKTTLSLTIQTELMRKAARFGVVLGENAPQTCRSAIQ
ASMQNLNTLIDGECAKTTFNDLLDYLHQEKIWVYTPHGQLHELPQGATVV
DFAYSASLFLGNHAVGAKVDGEIKPLSTPLVSGQVIEIITDVLATPNPDW
LSFINTQKARRALQHVLKDQDIEEQRLVGAQALSRALKLFNRSINDLSDA
DWLDLLQWRHIDNKDALFEQIAVGDLLPQLVANHLFANDKHPRAENSDRL
IQGTEGIDVKYAHCCNPILGDPIQGHLTRRGLIVHRIRCHNLLHEQHLHP
ENIMPLQWKADDVDDVRFTAYLAIYMAMNDEQVSDLIYQCRKNNAGVEMV
HSNEQRTFVNIVVNNRKHIAKVIRDLRMHYGFPRIERLDAPAPQMEISKV
S

Preferably, the amino acid sequence of A. baumannii SpoT enzyme comprises, consists essentially of, or consists of SEQ ID NO: 1.

In certain embodiments, interactions of said candidate compound to one or more amino acid residues of a region on the surface of the protein defined by amino acid residues: Arg45, Lys46, Ser47, Tyr51, His54, His78, Asp79, Ser113, Lys140, Asp143, Asn147, Thr150, Ala153, and Lys158 of the SpoT amino acid sequence as defined by an amino acid sequence with at least 70% sequence identity to the amino acid sequence of SEQ ID NO: 1 indicate that the candidate compound is a modulator of SpoT hydrolase activity. In preferred embodiments, interactions of said candidate compound to one or more amino acid residues of a region on the surface of the protein defined by any one or more of the following amino acid residues: Arg45, Lys46, Ser47, Tyr51, His54, His78, Asp79, Ser113, Lys140, Asp143, Asn147, Thr150, Ala153, and Lys158 of the SpoT amino acid sequence as defined by an amino acid sequence with at least 75%, preferably at least 80%, preferably at least 85%, preferably at least 90%, preferably at least 95% sequence identity to the amino acid sequence of SEQ ID NO: 1 indicate that the candidate compound is a modulator of SpoT hydrolase activity. In further preferred embodiments, interactions of said candidate compound to any one or more amino acid residues of a region on the surface of the protein defined by any one or more of the following amino acid residues: Arg45, Lys46, Ser47, Tyr51, His54, His78, Asp79, Ser113, Lys140, Asp143, Asn147, Thr150, Ala153, and Lys158 of the SpoT amino acid sequence as defined by an amino acid sequence that comprises, consists essentially of, or consists of SEQ ID NO: 1 indicate that the candidate compound is a modulator of SpoT hydrolase activity. The term “region on the surface of the protein” as used herein intends to refer to a surface patch that defines a binding site which involves the residues that are listed with respect to said region.

Optionally, the method comprises assessing whether the candidate compound interacts with at least 2, at least 3, at least 4, at least 5, at least 6, at least 7, at least 8, at least 9, at least 10, at least 11, at least 12, at least 13, or all (i.e. at least 14) amino acid residues of the group consisting of Arg45, Lys46, Ser47, Tyr51, His54, His78, Asp79, Ser113, Lys140, Asp143, Asn147, Thr150, Ala153, and Lys158 as defined by SEQ ID NO: 1. In such embodiments, the candidate compound is considered an A. baumannii SpoT enzyme modulator upon at least 2, at least 3, at least 4, at least 5, at least 6, at least 7, at least 8, at least 9, at least 10, at least 11, at least 12, at least 13, or all (i.e. at least 14) amino acid residues of the group consisting of Arg45, Lys46, Ser47, Tyr51, His54, His78, Asp79, Ser13, Lys140, Asp143, Asn147, Thr150, Ala153, and Lys158 as defined by SEQ ID NO: 1.

In certain embodiments, interaction of the candidate modulator with any one or more of the group of amino acids consisting of Arg45, Lys46, Ser47, Tyr51, His54, His78, Asp79, Ser113, Lys140, Asp143, Asn147, Thr150, Ala153, and Lys158 as defined by SEQ ID NO: 1 indicates that the candidate modulator is an inhibitor of A. baumannii SpoT hydrolase activity. In alternative embodiments, interaction of the candidate modulator with any one or more of the group of amino acids consisting of Arg45, Lys46, Ser47, Tyr51, His54, His78, Asp79, Ser113, Lys140, Asp143, Asn147, Thr150, Ala153, and Lys158 as defined by SEQ ID NO: 1 indicates that the candidate modulator is an activator of A. baumannii SpoT hydrolase activity.

Optionally, the screening method described herein may further comprise a step of determining a score of the candidate compound to modulate A. baumannii SpoT activity, preferably A. baumannii SpoT hydrolase activity, based on the number of interactions with Arg45, Lys46, Ser47, Tyr51, His54, His78, Asp79, Ser113, Lys140, Asp143, Asn147, Thr150, Ala153, and Lys158 as defined by SEQ ID NO: 1. In such embodiments, an increasing amount of interactions with said amino acid residues results in a more favourable score for the candidate compound. The score may be expressed as an absolute value and/or as a relative value compared to one or more reference A. baumannii SpoT modulator molecules. In an illustrative embodiment, the score may be a positive integer that is a sum of the number of interactions between the amino acid residues described herein and the candidate compound. In an alternative illustrative embodiment, the score may be a percentage, wherein 0% indicates no interaction(s) between the candidate compound and the A. baumannii SpoT protein, and 100% indicates an interaction with each of the amino acid residues described herein that are indicated to form, or be part of, the relevant portion of the A. baumannii SpoT surface region as defined herein. It is evident that a candidate compound with a higher score, said score being linearly correlated to the amount of interactions, indicates a higher likelihood of a candidate compound to be a strong modulator (e.g. inhibitor) of the A. baumannii SpoT protein when compared to a candidate compound with a lower score.

Optionally, the screening method may use as input, or prerequisite, that the candidate compounds interact with the interface of the Core domain and the regulatory CTD region. For example, the method may comprise an initial step where only candidate compounds are retained that are known to bind, considered to bind, or predicted to bind, the interface between the Core domain and the regulatory CTD region.

Optionally, the method further comprises comparing the conformational state of A. baumannii SpoT before and after said candidate compound binds to A. baumannii SpoT, wherein a change in conformational state is indicative for the candidate compound to be a bona fide modulator of A. baumannii SpoT hydrolase activity, preferably wherein the general conformational state of A. baumanni SpoT after candidate binding differs from the atomic coordinates presented in Table 1 by a root mean square deviation (RMSD) of residue over protein backbone atoms by no more than 3 Å, more preferably 2 Å, yet more preferably 1 Å, most preferably wherein the general conformational state of A. baumannii SpoT after candidate binding is the conformational state characterized by the subset of atomic coordinates of Table 1 that define the A. baumannii SpoT enzyme.

A “conformational change” as described herein is to be understood as a change in the three-dimensional shape of a molecule, in the context of the present invention A. baumannii SpoT. A conformational change may be induced by numerous factors including the non-limiting examples of temperature, pH, voltage, light, ion concentration, post translational modification or binding to a second molecule. The conformational change as described in the current application is a consequence, either directly or indirectly, of binding to a modulator molecule. A protein may display different functions and/or engage in distinct interactions depending on its conformation. In light of the current invention, the conformational state may impact, and preferably impacts, the hydrolase activity level of A. baumannii SpoT. In these preferred embodiments, the conformational state of A. baumannii is a conformation state that is characterised by a reduced, or even completely lack of hydrolase activity by the enzyme. In certain embodiments, specific conformations partially or even completely inhibit hydrolase and/or synthetase activity. In alternative embodiments, specific conformations cause an upregulation of the hydrolase and/or synthetase activity. When “stabilization” of a conformational state is described in the context of the current invention upon binding an A. baumannii SpoT modulator, it is intended that the SpoT protein adopts a particular state such as but not limited to an open or closed state for at least the time window wherein candidate compound-SpoT interaction is occurring.

In certain embodiments, the method comprises detection of any atomic coordinates that are different after binding of the candidate A. baumannii SpoT modulator from the atomic coordinates characterizing the bound active conformational state of A. baumannii SpoT shown in Table 1.

Preferably, the method is a method for identifying compounds that inhibit A. baumannii SpoT hydrolase activity when compared to a reference condition wherein the compound is not present. Preferably, the method is a method for identifying compounds that inhibit A. baumannii SpoT hydrolase activity by at least 30%, more preferably by at least 40%, more preferably by at least 50%, more preferably by at least 60%, more preferably by at least 70%, more preferably by at least 80%, more preferably by at least 90%, more preferably by at least 95% when compared to a reference condition wherein no compound is present. Optionally, the method is a method for identifying compounds that fully inhibit A. baumannii SpoT hydrolase activity (i.e., inhibition of 100%, or below any detectable activity levels).

Optionally, the method is a method for identifying compounds that increase A. baumannii SpoT hydrolase activity when compared to a reference condition wherein the compound is not present. Preferably, the method is a method for identifying compounds that increase A. baumannii SpoT hydrolase activity by at least 30%, more preferably by at least 40%, more preferably by at least 50%, more preferably by at least 60%, more preferably by at least 70%, more preferably by at least 80%, more preferably by at least 90%, more preferably by at least 95% when compared to a reference condition wherein no compound is present. Optionally, the method is a method for identifying compounds that increase A. baumannii SpoT hydrolase activity (i.e., inhibition of 100%, or below any detectable activity levels). In certain embodiments, the method is a method for identifying compounds that increase A. baumannii SpoT hydrolase activity by at least 1.5 fold, preferably at least 2 fold, more preferably at least 5 fold, most preferably at least 10 fold.

In certain embodiments, the method further comprises testing of the ability of the candidate compounds for modulating A. baumannii SpoT hydrolase activity. In certain embodiments, the method comprises in vitro and/or in vivo testing of the ability of the candidate compounds for inhibiting or increasing A. baumannii SpoT hydrolase activity, preferably inhibiting A. baumannii SpoT hydrolase activity. In certain embodiments, the testing of the candidate compounds involves testing of said compound in competition with one or more natural A. baumannii SpoT substrates such as (p)ppGpp.

By means of an illustrative example, in vitro testing of the hydrolase activity of A. baumannii SpoT in presence of an candidate A. baumannii SpoT hydrolase-modulating compound can comprise contacting said candidate compound with recombinant A. baumannii SpoT protein and measuring removal of the 3′ pyrophosphate moiety from (p)ppGpp (i.e. monitoring the hydrolysis reaction mediated by A. baumannii SpoT). Similar experimental conditions can be devised for in vivo activity testing. Methods for assessing a plethora of different enzymatic activities are known in the art (Ou et al., Annu Rev Anal Chem, 2018).

In certain embodiments, the screening methods described herein are computer-implemented methods. In further embodiments, the computer comprising an inputting device, a processor, a user interface, and an outputting device. In such embodiment, the said method may comprises the steps of:
a) generating a three-dimensional structure of the atomic coordinates of Table 1, or any subset thereof as described in the present disclosure;
b) fitting the structure of step a) with the structure of a candidate compound by computational modeling;
c) selecting a ligand that possesses energetically favorable interactions with the structure of step
a).

In certain embodiments, the method further comprises selection of ligands that possess multiple energetically favorable interactions with said three-dimensional structure in favor of ligands that possess one energetically favorable interaction with said three-dimensional structure. In certain embodiments, the three-dimensional structure is generated using the atomic coordinates from at least one subset of atomic coordinates of Table 1 as described herein. In alternative embodiments, the three-dimensional structure is generated using the complete list of atomic coordinates presented in Table 1.

The term “energetically favorable interaction” as used herein is envisaged any interaction with interaction energies <0 kJ/mol. Alternatively an energetically favorable interaction may be expressed as an interaction having a negative Gibbs free energy (AG) value. Since a protein-ligand association extent is correlated to the magnitude of a negative AG, AG can be regarded as determinant for the stability of the protein-ligand complex under investigation, or, alternatively, the binding affinity of a ligand to a given acceptor, in the context of the current specification the A. baumannii SpoT enzyme. Free energy is a function of the states of a system and, as thus, AG values are defined by the initial and final thermodynamic state, regardless of any intermediates states. The concept of energetically favorable interactions is known to a person skilled in the art (Du et al., Int J Mol Sci, 2016).

In certain embodiments, the method comprises superimposing the generated three-dimensional structure of the SpoT enzyme or SpoT-ppGpp complex with the structure of the candidate compound. In further embodiments, the method comprises selecting from a collection of distinct structure-candidate compound superimposed orientations a most favorable orientation of said structure with said candidate compound. Hence, in certain embodiments, the method comprises docking modeling or molecular docking. In certain embodiments, the method comprises a computer-implemented step of proposing candidate structure modifications to further increasing the number of favorable interactions with the generated three-dimensional structure. In yet further embodiments, the method comprises ranking an obtained collection of candidate compounds based on the number of favorable interactions they engage in with the generated three-dimensional structure, wherein candidate compounds with a higher number of favorable interactions are ranked higher than candidate compounds with fewer favorable interactions.

The terms “docking modeling” and “molecular docking” are indicative for one or more quantitative and/or qualitative analyses of a molecular structure based on structural information and interaction models. Modeling may refer to any one of numeric-based molecular dynamic models, interactive computer graphic models, energy minimization models, distance geometry, molecular mechanics models, or any structure-based constraints model. These illustrative molecular modeling approaches may be employed to the atomic coordinates or a subset of atomic coordinates as described herein in Table 1 to obtain a range of three-dimensional models and to investigate the structure of any binding sites, such as the binding sites of candidate A. baumannii SpoT modulators. Modeling methods and tools have been developed to design or select chemical molecules that have a complementarity to particular target regions, in the context of the invention a particular target region of A. baumannii SpoT. In certain embodiments, the chemical molecule, i.e. the candidate compound has a stereochemical complementarity to said target regions. In certain embodiments, the candidate compound has a general structural similarity to ppGpp. Stereochemical complementarity refers to a scenario wherein there are a number of energetically favorable contacts between the candidate compound and (the target region of) A. baumannii SpoT. A skilled person appreciates that if a certain number of energetically favorable interactions are sufficient to modulate A. baumannii SpoT activity, and that it is thereby not a precondition that all the key amino acid residues as described herein are engaged in an energetically favorable interaction. Non-limiting examples of software programs suitable for conducting molecular docking analysis have been described in detail in the art (Pagadala et al., Biophys Rev, 2017).

Any computer system or any computer-implemented method relying on a computer system described herein may further comprise means for machine learning of said device to predict candidate A. baumannii SpoT modulators, such as hydrolase inhibitors, and/or score said modulators based on input of a reference set of candidate compounds by a user, or based on date generated from earlier fitting and/or selection steps of candidate modulators. The combination of machine learning models for in silico screening and prediction of enzyme binding molecules or modulators is known in the art, and therefore also envisaged by the current invention (Li, et al., Molecules, 2019). Non-limiting examples of machine learning models, i.e. machine learning algorithms include Linear regression, logistic regression, decision trees, support vector machines, naive Bayes, k-nearest neighbors (kNN), k-means, random forest, dimensionality reduction algorithms, and gradient boosting algorithms such as gradient boosting machine (GBM), XGBoost, LightGBM, and CatBoost.

In certain embodiments, the method comprises selecting a candidate compound that can bind to at least 1 amino acid residue, preferably more than 1 amino acid residue of the generated three-dimensional structure without steric interference. The terms “steric interference”, “steric hindrance”, and “steric effects” are known to a person skilled in the art. Steric interference or alternatively referred to as steric hindrance is a consequence of a steric effect, and indicates the slowing of chemical reactions due to steric bulk.

Further aspects herein relate to an in vitro method for identifying a compound which specifically modulates A. baumannii SpoT hydrolase activity comprising the steps of:
a) providing a candidate compound;
b) providing the A. baumannii SpoT protein or SpoT-ppGpp complex;
c) contacting said candidate compound with said SpoT protein or SPoT-ppGpp complex;
d) determining the hydrolase activity of A. baumannii SpoT in the presence and absence of said candidate compound; and
e) identifying said candidate compound as a compound which modulates A. baumannii SpoT hydrolase activity if a change in activity is detected.

A skilled person appreciates that the expression “specifically modulates” indicates that the compound acts on the A. baumannii SpoT hydrolase activity in a manner that is directly altering the enzymatic hydrolase activity. The expression therefore excludes compounds that may modulate the A. baumannii SpoT hydrolase activity indirectly, such as for example impacting the viability of the organism as a whole, or impacting SpoT hydrolase protein expression levels. Thus preferably, the specific modulation of the hydrolase activity of the SpoT protein occurs by direct binding of the candidate compound to said SpoT protein or SpoT-ppGpp complex.

An illustrative method to assess hydrolase activity is described above. In certain embodiments, the method comprises further selecting additional candidate compounds based on common structural features from a database. In certain embodiments, recombinant A. baumannii SpoT protein is used in the methods described herein. Means and methods to produce and purify recombinant protein have been described in detail in the art (inter alia in Grasslund et al., Nat Methods, 2011).

In certain embodiments, the A. baumannii SpoT protein (optionally in complex with ppGpp) is characterized by an amino acid sequence that has at least 70% sequence identity to SEQ ID NO: 1. In preferred embodiments, the A. baumannii SpoT protein (optionally in complex with ppGpp) is characterized by an amino acid sequence that has at least 80% sequence identity, more preferably at least 85% sequence identity, more preferably at least 90% sequence identity, even more preferably at least 95% sequence identity to SEQ ID NO:1. In most preferred embodiments, the A. baumannii SpoT protein (optionally in complex with ppGpp) comprises, consists essentially of, or consists of SEQ ID NO: 1. In preferred embodiments, the A. baumannii SpoT protein and/or A. baumannii SpoT-ppGpp complex is defined by the atomic coordinates of Table 1.

In certain embodiments, the method further comprises immobilization of the A. baumannii SpoT protein, SpoT-ppGpp complex, or the candidate compound on a solid surface. In further embodiments, the method comprises a step of washing away excess A. baumannii SpoT protein, SpoT-ppGpp complex, or excess candidate compound prior to determining the hydrolase activity. In certain embodiments, the method comprises detecting a change in hydrolase activity by colorimetry or spectrophotometry. In certain embodiments, a change of activity is considered as an increase of hydrolase activity of the A. baumannii SpoT protein by at least 10%, preferably 25%, preferably 50%, preferably 75%, preferably 100% in presence of said candidate compound when compared to the hydrolase activity when the enzymatic activity of said A. baumannii SpoT protein is assessed in absence of any (candidate) compound. Alternatively, a change of activity is considered as an increase of hydrolase activity of the A. baumannii SpoT protein by at least 1.5 fold, preferably at least 2 fold, more preferably at least 5 fold, most preferably at least 10 fold. In alternative embodiments, a change of activity is considered as a decrease of hydrolase activity of the A. baumannii SpoT protein by at least 10%, preferably 25%, preferably 50%, preferably 75%, preferably 100% in presence of said candidate compound when compared to the hydrolase activity when the enzymatic activity of said A. baumannii SpoT protein is assessed in absence of any (candidate) compound. Alternatively, a change of activity is considered as a decrease of hydrolase activity of the A. baumannii SpoT protein by at least 1.5 fold, preferably at least 2 fold, more preferably at least 5 fold, most preferably at least 10 fold. In certain embodiments, the method identifies candidate compounds capable of inhibiting the hydrolase activity to such an extent that no SpoT hydrolase activity can be detected by methods described in the state of the art. In alternative embodiments, the method identifies candidate compounds capable of stimulating the hydrolase activity.

A further aspect of the invention relates to the use of the crystal structure of the A. baumannii SpoT-protein or SpoT-ppGpp complex as defined by the atomic coordinates presented in Table 1, or a subset thereof as described herein, or atomic coordinates which deviate from those in Table 1, or a subset thereof, by RMSD over protein backbone atoms by no more than 3 Å for designing and/or identifying a compound which modulates, preferably partially or completely inhibits A. baumannii SpoT hydrolase activity.

The term “crystal structure” as used herein is a three-dimensional description of ordered arrangements or structures of elements such as atoms, ions, or molecules in a crystalline material. Crystal structure refers to a protein crystal structure obtained by protein crystallography, the process of forming a protein crystal by experimentation, unless stated otherwise. In a typical protein crystallization process, proteins are dissolved in an aqueous environment comprising a sample solution until supersaturation is obtained. Different approaches have been described in detail in the art and include as non-limiting examples vapor diffusion, batch, microdialysis and liquid-liquid diffusion. Once a protein crystal is obtained, different techniques such as X-ray diffraction, cryo-electron microscopy, or nuclear magnetic resonance are suitable to determine the protein crystal structure. The term “supersaturation” refers to a condition of a solution that contains more of a dissolved material than can be dissolved by the solvent under normal conditions and has been defined in the art as a non-equilibrium condition in which some quantity of the macromolecule in excess of the solubility limit, under specific chemical and physical conditions, is nonetheless present in solution (McPherson and Gavira, Struct Biology Commun, 2014). Protein crystals thus also compose a large amount of solvent molecules such as the non-limiting example of water. Due to the different methodologies for preparing a protein crystal, these crystals further comprise a varying range of buffers, salts, small binding proteins, and precipitation agents which can vary substantially in concentration. Typical crystals have a size of between 20 μm to multiple mm. A crystal optimal for X-ray diffraction analysis is ideally free of cracks and other defects.

In a further aspect of the invention, the inventors have found that compounds such as small molecules that interact with the A. baumannii SpoT protein via the interface between the Core domain and the regulatory domain are of particular interest to act as A. baumannii SpoT modulators. Without wishing to be bound by theory, it is hypothesized that pseudo-SYNTH, ZFD and RRM all subtly tune the HD activity of A. baumannii SpoT protein up or down by modulating its interactions with the Core. The presence of the Core and its crosstalk with the HD/pseudo-HD domain likely constitutes a universal structural requirement for the efficient stabilization of the active states of long RSHs.

Thus, in certain embodiments, the candidate A. baumannii SpoT protein modulator is a compound that binds to the interface of the Core domain and the regulatory domain.

Yet a further aspect of the invention concerns a computer system comprising a database containing the atomic coordinates as presented in Table 1, or a subset thereof as described herein, stored on a computer readable storage medium, and a user interface to view the information. Also intended are data processing apparatuses, devices, and systems comprising a database containing the atomic coordinates as presented in Table 1, or a subset thereof as described herein stored on a computer readable storage medium, and a user interface to view the information. Models and atomic coordinates as disclosed herein are typically stored on a machine-readable, or computer-readable medium which are known in the art and include as non-limiting examples magnetic or optical media and random-access or read-only memory, including tapes, diskettes, hard disks, CD-ROMs and DVDs, flash drives or chips, servers and the internet. In certain embodiments, the computer system comprises means for carrying out the methods as described herein. In certain embodiments, the computer system further comprises an input device to receive instructions from an operator. In certain embodiments, the computer system comprises and/or is connected to a remote data storage system, wherein the remote data storage system is located at a geographic location different from the location of the user interface to view the information. Said data storage system may be located in a network storage medium such as the internet, providing remote accessibility. In certain embodiments, the database comprised in the computer system is encrypted. In certain embodiments, the computer system has access to at least one database of compound structures, and a user can by appropriately instructing said computer system access said at least one database of compound structures. In certain embodiments, the compound, list of compounds, or compound database (also known as compound library) is loaded into the computer system by the operator. In alternative embodiments, the compound, list of compounds, or compound database is accessible by the computer system from a medium different than said computer system. In certain embodiments, the computer system comprises a processing unit to assess the degree of fit between any compound molecule loaded into the computer system and A. baumannii SpoT protein and/or SpoT-ppGpp complex. Also intended is a computer-readable storage medium comprising instructions which, when executed by a computer, causes the computer to carry out any one of the methods disclosed herein.

A further aspect relates to the use of a computer system as described herein for designing and/or identifying a compound (ligand) which modulates A. baumannii SpoT activity. In certain embodiments, the use of said computer system is achieved by user input commands. In certain embodiments, the computer system comprises means to select candidate A. baumannii SpoT modulators from a list of compounds, or a compound library. In certain embodiments, the computer system comprises means to select (a) candidate compound(s) and proposing structural changes to the at least one candidate compound to further increase the number of energetically favorable interactions between said compound and A. baumannii SpoT and/or means to select (a) candidate compound(s) and proposing structural changes to the at least one candidate compound to reduce or eliminate structural interference between said candidate modulator and one or more residues of A. baumannii SpoT defined by the atomic coordinates in any one of Table 1. When using a computer system as described herein, the user searching for A. baumannii SpoT modulators, which may or may not be the operator of the computer is provided by an optionally printed list of candidate A. baumannii SpoT modulators, preferably A. baumannii SpoT hydrolase inhibitors. The computer system provides the user with one or more candidate A. baumannii SpoT modulators, preferably A. baumannii SpoT hydrolase inhibitors. In certain embodiments, the computer system is configured to be exclusively suited for providing the user with candidate compounds that inhibit A. baumannii SpoT hydrolase activity. In alternative embodiments, the computer system can be used to only provide the user with candidate compounds that upregulate (i.e. increase) A. baumannii SpoT hydrolase activity. In alternative embodiments, the computer system is used for designing and/or identifying an allosteric A. baumannii SpoT modulator. In certain embodiments, the computer system is used to provide a visual representation, i.e. an image of the three-dimensional structure of A. baumannii SpoT, optionally during interaction with the candidate A. baumannii SpoT compound. In certain embodiments, a list of candidate A. baumannii SpoT modulators is generated and stored, optionally sorted according to a scoring system as described herein, in an electronic file.

Yet a further aspect of the invention is directed to a crystal of A. baumannii SpoT protein and/or SpoT-ppGpp complex, comprising a structure characterized by the atomic coordinates as presented in Table 1 or a subset thereof as described herein. A skilled person appreciates that the crystal structure characterised by the atomic coordinates as presented in Table 1 correspond to the SpoT-ppGpp complex, and therefore represents an active, bound state of the enzyme. Optionally, the crystal is obtained by crystallizing a protein comprising SEQ ID NO: 1, or by cristallizing A. baumanni SpoT protein as defined by SEQ ID NO: 1. Optionally, the crystal is obtained by crystallization of A. baumannii SpoT protein in a solution using a space group p21 21 21, and a unit cell: 128.791 133.761 211.328 90.00 90.00 90.00 and supplementing ppGpp to the solution prior to crystal harvesting, preferably prior to crystal harvesting at 50 mM. Optionally, the solution comprises, consists essentially of, or consists of 0.85 M Sodium citrate tribasic dihydrate, 0.1 M Tris pH 8.0, and 0.1 M Sodium chloride.

Any crystal structure disclosed herein is said to be characterized by, or conform to, or substantially conform to, a set or subset of atomic coordinates when a structure, or a substantial fragment of a structure falls within the limit RMSD value as disclosed herein. In a certain embodiment, at least 70%, preferably at least 75%, more preferably at least 80%, even more preferably at least 90%, yet more preferably at least 95% of the crystal structure has the recited RMSD value. In certain embodiments, “substantially conform to” further refers to atoms of amino acid side chains. In this context, common amino acid side chains are side chains that are common between the structure substantially conform to a structure with particular atomic coordinates and structures being defined by said atomic coordinates of Table 1. In one embodiment, the coordinates on the ppGpp binding within the coordinates of the A. baumannii SpoT protein presented in Table 1 can be used to identify the binding pocket of said stabilized conformation (atomic coordinates corresponding to the ppGpp molecule are those indicated by the identifier “G4P”). Alternatively, the coordinates could be removed for ease of modelling new molecules or agents into the A. baumannii SpoT protein conformation.

A further aspect of the invention is directed to a computer system, intended to generate three dimensional structural representations of an A. baumannii SpoT protein and/or SpoT-ppGpp complex, complexes of A. baumannii SpoT protein with binding compounds or modulators to analyze or optimize binding (i.e. for analyzing or optimizing binding) of compounds or modulators to said A. baumannii SpoT protein and/or SpoT-ppGpp complex, the system containing computer-readable data comprising one or more of:
(a) the coordinates of the A. baumannii SpoT protein structure listed in Table 1 (i.e. Table 1 excluding those coordinates having the identifier “G4P”), optionally varied by a root mean square deviation of residue backbone atoms of not more than 3 Å, or selected coordinates thereof,
(b) the coordinates of the A. baumannii SpoT-ppGpp complex structure listed in Table 1, optionally varied by a root mean square deviation of residue backbone atoms of not more than 3 Å, or selected coordinates thereof,
(c) the coordinates of a candidate binding compound or modulator generated by interpreting X-ray crystallographic data, cryo-EM, or NMR data by reference to the coordinates of the A. baumannii SpoT protein structure and/or SpoT-ppGpp complex structure, listed in Table 1, optionally varied by a root mean square deviation of residue backbone atoms of not more than 3 Å, or selected coordinates thereof, and
(d) structure factor data derivable from the coordinates of (a), (b) or (c).

In certain embodiments, the computer system comprises data comprising any combination of (a), (b), (c), or (d). In further embodiments, the user is able to adjust, remove, or add further data to the computer system. In certain embodiments, the computer system is able to receive additional data, adjust data, or remove data pertaining to (a), (b), (c), or (d). In certain embodiments, the user is able to access synthesis protocols of compounds or modulators through the computer system. In certain embodiments, the computer system directs the user to a synthesis protocol.

Optionally, the herein described computer system compares the atomic coordinates of (a) and (c), and wherein when a sterical conflict is detected the candidate compound or modulator is not considered a suitable A. baumannii SpoT protein modulator. Optionally, the herein described computer system compares the atomic coordinates of (a) and (c), an wherein when no sterical conflict is detected the candidate compound or modulator is considered a suitable A. baumannii SpoT protein modulator.

A different aspect of the invention relates to a computer-readable storage medium, comprising a data storage material encoded with computer readable data, wherein the data comprises one or more of
(a) the coordinates of the A. baumannii SpoT protein structure (i.e. Table 1 excluding those coordinates having the identifier “G4P”), listed in Table 1, optionally varied by a root mean square deviation of residue backbone atoms of not more than 3 Å, or selected coordinates thereof,
(b) the coordinates of the A. baumannii SpoT-ppGpp complex structure listed in Table 1, optionally varied by a root mean square deviation of residue backbone atoms of not more than 3 Å, or selected coordinates thereof,
(c) the coordinates of a candidate binding compound or modulator generated by interpreting X-ray crystallographic data, cryo-EM or NMR data by reference to the coordinates of the Rel enzyme structure, listed in Table 1, optionally varied by a root mean square deviation of residue backbone atoms of not more than 3 Å, or selected coordinates thereof, and
(d) structure factor data derivable from the coordinates of (a), (b) or (c).

In certain embodiments, the computer readable data is encrypted and requires authentication or authorization credentials from a user or second computer-readable storage system for a computer system to be able to access said data. In certain embodiments, the computer-readable storage medium is a physical storage medium. In alternative embodiments, the computer-readable storage medium is a non-physical storage medium or a storage medium perceived to be a non-physical storage medium (i.e. a cloud based storage medium).

Another aspect of the invention relates to a computer-readable storage medium comprising a data storage material encoded with a first set of computer-readable data comprising a Fourier transform of at least a portion of the structural coordinates of the A. baumannii SpoT protein or SpoT-ppGpp complex listed in Table 1, optionally varied by a root mean square deviation of residue backbone atoms of not more than 3 Å, or selected coordinates thereof, which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data. Fourier transformation in the context of the invention is to be interpreted as the application of a molecular-replacement approach.

As envisaged herein by the term “Fourier transform”, the three-dimensional transformation of a molecular model is calculated in a first step. Subsequently, the weighed reciprocal lattice is rotated according to the calculated transformation. Fourier transformation in molecular biology, and more specifically structure biology, has been described in the art (Rabinovich et al., Acta crystallographica section D biological crystallography, 1998). In certain embodiments, the X-ray diffraction pattern of a molecule or molecular complex of unknown structure is obtained by an apparatus operably coupled to said computer storage medium. In alternative embodiments, the X-ray diffraction pattern of a molecule or molecular complex of unknown structure is inputted to said computer-readable storage medium by user instructions. In yet alternative embodiments, the X-ray diffraction pattern of a molecule or molecular complex of unknown structure is retrieved by a computer system comprising the computer-readable storage medium from a public (accessible) database.

In certain embodiments, the computer system or computer-readable storage medium as described herein further comprises a database containing information on the three dimensional structure of candidate compounds or modulators which are small molecules. In certain embodiments, the computer system or computer-readable storage medium further comprises a means to retrieve information from public information databases on the three dimensional structure of candidate compounds or modulators, which preferably are “small” molecules as defined herein that partially or completely inhibit the hydrolase activity of A. baumannii SpoT, including the non-limiting examples of PubChem (https://pubchem.ncbi.nlm.nih.gov), the Zinc database (https://www.zinc.docking.org), and/or MolPort (https://www.molport.com). In certain embodiments, the computer system further generates information indicating which list or subset of atomic coordinates of Table 1 shows, or is predicted to show, the highest number of energetically favorable interactions with any candidate modulator assessed by said computer system. In certain embodiments the user receives an automatically generated list of candidate compounds ranked according to the number of energetically favorable interactions with the A. baumanni SpoT protein as defined by each list or subset of atomic coordinates of Table 1. In further embodiments the computer system provides the user with a number of common structural groups any combination of candidate modulator, or even hydrolase inhibitor, may be differentiated by.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples. The following specific experimental examples are provided in support of the claimed invention but are not to be seen as limiting the scope of the invention.

### EXAMPLES

### 1. A. baumannii SpoT_{Ab} is a Monofunctional Hydrolase Long RSH

Lack of conservation of active site residues critical for SYNTH activity suggest that Moraxallaceae SpoT enzymes have—like RelA—undergone subfunctionalisation to become monofunctional long RSHs. Like RelA's pseudo-HD domain, the SYNTH domain region has been retained in Moraxallaceae as a presumably non-catalytic pseudo-SYNTH domain, suggesting it retains some function in stabilisation or allosteric regulation of the HD domain. To probe the hydrolysis function of A. baumannii SpoT (SpoT_{Ab}) in live cells, we leveraged SpoT's hydrolytic activity being crucial for controlling the cellular levels of (p)ppGpp produced by RelA, which makes SpoT conditionally essential in the relA⁺Escherichia coli (Xiao et al., J Biol Chem, 1991). We co-transformed a ppGpp⁰ (ΔrelA ΔspoT) E. coli strain with i) a pMG25-based plasmid driving the IPTG-inducible expression of spoT_{Ab} under the control of PA 1/O4/O3 promoter and ii) a pMR33 derivative for arabinose-inducible expression of relA_{Ec} under the control of PBAD. While expression of the (p)ppGpp synthetase RelA_{Ec} strongly inhibited the growth of ppGpp⁰ E. coli, the growth was completely restored upon the ectopic co-expression of SpoT_{Ab}, demonstrating that SpoT_{Ab} is HD-active in the surrogate E. coli host.

Next, we used our dual plasmid co-expression system to probe the (p)ppGpp synthetase activity of SpoT RSHs. ppGpp®⁰ E. coli is auxotrophic for eleven amino acids, and (p)ppGpp synthetase activity of SpoT_{Ec} is essential for growth of ΔrelA E. coli on minimal medium (Xiao et al., J Biol Chem, 1991). Unlike the SYNTH-active SpoT_{Ec}, SpoT_{Ab} failed to promote the growth of ppGpp⁰ E. coli on M9 minimal medium, confirming that SpoT_{Ab} is SYNTH-inactive. Taken together, these results demonstrate that SpoT_{Ab} is a specialised monofunctional long RSH that lacks the ability to synthesise (p)ppGpp.

### 2. Full-Length SpoT_{Ab} has a Compact Mushroom-Like T-Shaped Structure

To gain insight into the molecular workings of SpoT, we solved an X-ray structure of full length catalytically-active SpoT_{Ab} in a ppGpp-bound state at 2.9 Å resolution.

The amino acid sequence SEQ ID NO: 1 of A. baumannii SpoT protein was used for the crystallisation experiments. This protein was crystallised at 4° C. in 0.85 M Sodium citrate tribasic dihydrate, 0.1 M Tris pH 8.0, and 0.1 M Sodium chloride (Space group: p2₁ 2₁ 2₁; Unit cell: 128.791 133.761 211.328 90.00 90.00 90.00). For soaking, ppGpp was supplemented to the solution prior to crystal harvesting at 50 mM together with the cry-protectant solution.

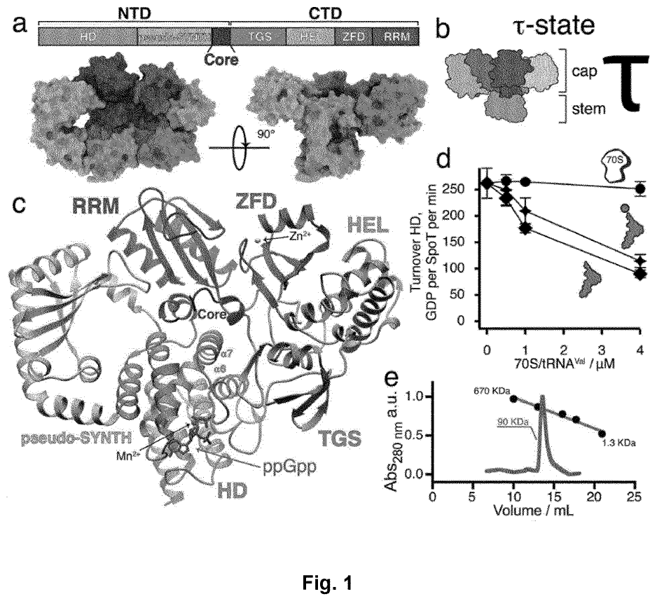

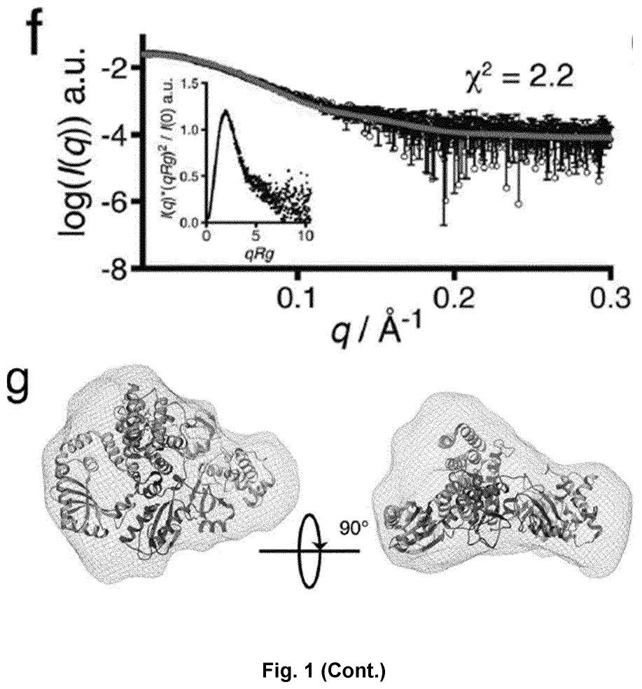

The structure revealed a multi-domain architecture strikingly different to that observed earlier for ribosome bound long RSHs Rel and RelA (Arenz et al., Nucleic Acids Res, 2016; Brown et al., Nature 2016; Loveland et al., Elife 2016; Pausch et al., Cell Rep, 2020) (FIG. 1a-c). The HD, SYNTH, TGS, HEL, ZFD and RRM domains of SpoT_{Ab} form a mushroom-like tau (τ)-shaped quaternary structure (FIG. 1a-c). In this arrangement, pseudo-SYNTH, TGS, HEL, ZFD and RRM domains all lie in a single plane and form a compact disc-like structure that forms the “cap” of the “mushroom” (FIG. 1b). A helix-turn-helix sub-domain (residues 334 to 379) that provides the transition between the NTD and CTD regions, lies at the “Core” of the “cap” and seemingly mediates interactions among all domains of the enzyme. Such an arrangement suggests that the Core—which is disordered in Rel/RelA structures—stabilises the disc-like “cap” of SpoT (FIG. 1c). Moreover, the Core provides the HD domain with a physical link to each domain of SpoT_{Ab}. Finally, the HD protrudes from the plane of the “cap” in the opposite direction of the C-terminal RRM domain, forming the “stem” of the protein structure (FIG. 1b-c).

The τ-shaped structure of SpoT_{Ab} suggests a possible structural mechanism for the auto inhibition of SYNTH activity by the regulatory CTD both in Rel (Mechold et al., J Bacteriol, 2002; Takada et al., Nucleic Acids Res, 2021) and RelA (Svitil et al., J Biol Chem, 1993; Turnbull, Front Microbiol, 2019). While the SYNTH and TGS domains are sequestered in the “cap”, the HD hydrolase stands out unconfined and primed for (p)ppGpp hydrolysis. The TGS domain, which in the case of amino acid starvation sensors Rel and RelA specifically engages the deacylated tRNA CCA-3′ end at the A site (Brown et al., Nature 2016; Loveland et al., Elife 2016; Pausch et al., Cell Rep, 2020; Winther et al., Mol Cell, 2018), in the case of SpoT_{Ab} is partially trapped between the HD, HEL and ZFD domains. While we do detect a mild inhibitory effect of tRNA on SpoT_{Ab} hydrolysis activity, the effect is insensitive to tRNA aminoacylation status, i.e. non-specific (FIG. 1d). This is in contrast to the HD activity of bifunctional E. coli SpoT (SpoT_{EC}), which was specifically inhibited by deacylated, but not aminoacylated tRNA (Richter, Gen Genet, 1980).

Our structure reveals that the sites from the ZFD and RRM domains that mediate rRNA recognition in Rel/RelA (Arenz et al., Nucleic Acids Res, 2016; Brown et al., Nature 2016; Loveland et al., Elife 2016; Pausch et al., Cell Rep, 2020; Winther et al., Mol Cell, 2018) are held in by the Core subdomain, suggesting that in the τ-shaped conformation the hydrolytically active (HD^{ON}) SpoT_{Ab} is incompatible with ribosome binding. In good agreement with this structural prediction, while the ribosome strongly suppresses the HD activity of Bacillus subtilis Rel (Rel_{Bs}) (Takada et al., Nucleic Acids Res, 2021), the addition of E. coli 70S has no effect on the hydrolysis activity of SpoT_{Ab} (FIG. 1d). Thus, our biochemical results suggest that SpoT_{Ab} is a ribosome-independent enzyme.

The τ-state of full-length monofunctional SpoT_{Ab}, enables auto-stimulation of the HD activity by the CTD via the Core domain, to regulate (p)ppGpp hydrolysis. The pseudo-SYNTH, ZFD and RRM all subtly tune the HD activity of SpoT_{Ab} up or down by modulating its interactions with the Core. The presence of the Core and its crosstalk with the HD/pseudo-HD domain likely constitutes a universal structural requirement for the efficient stabilization of the active states of long RSHs. In this sense, small molecules that interact with SpoT_{Ab} via the interface between the Core and the regulatory will inhibit the activity of the enzyme and could be used as starting point for drug design.

### 3. Shorter Intrinsically Disordered Regions (IDRs) in Monofunctional SpoT are Associated with Specialisation for Hydrolysis

The presence of intrinsically disordered regions (IDR) located at the α6-α7 loop, the Core subdomain and the linker between HEL/ZFD domains in long RSHs RelA and Rel has posed an experimental challenge for structural studies (Arenz et al., Nucleic Acids Res, 2016; Brown et al., Nature 2016; Loveland et al., Elife 2016; Pausch et al., Cell Rep, 2020). The molecular function of these flexible regions, unresolved in the structures, is unknown. Comparison between the well-structured SpoT_{Ab} in τ-state and partially unstructured ribosome bound RelA/Rel suggests that the unfolding of Core and HEL domains constitutes part of the conformational switch that positions TGS, ZFD and RRM domains to stimulate the synthesis activity of Rel/RelA upon recruitment to the ribosome.

The length of these disordered or flexible regions is on average shorter in monofunctional SpoT and much longer in the monofunctional RelA. Bifunctional Rels have interdomain IDRs of sizes between both monofunctional enzymes. The α6-α7 loop of the HD domain of SpoT[Hs] in particular is a third of the size of that of RelA, which, in turn, is twice longer than that of bifunctional Rel. The same pattern is observed for the other two IDRs: the Core subdomain and the region connecting HEL and ZFD domains. This is consistent with the significantly lower disordered propensity of the Core of SpoT_{Ab} compared to RelA_{Ab}. We speculate that these IDRs have evolved to stabilise either τ-(shorter IDRs) or elongated (longer IDRs) states of monofunctional SpoT[Hs] or RelA[hS], respectively, to tune the HD vs SYNTH output ratio.

### 4. SpoT_{Ab} is a Monomer

It was shown earlier that both Rel and RelA are prone to dimerization via the CTD, which would potentially serve to regulate their enzymatic activity (Pausch et al, Cell Rep, 2020; Gropp et al., J Bacteriol, 2001; Kaspy and Glaser, Front Microbiol, 2020; Yang and Ishiguro, Biochem Cell Biol, 2001). This idea is a subject of debate, with both genetic (Turnbull et al., Front Microbiol, 2019) and mass photometry (Takada et al., Nucleic Acids Res, 2021) experiments suggesting that the dimerization is unlikely to take place at physiologically relevant concentrations. Therefore, we used small-angle X-ray scattering (SAXS) coupled to size exclusion chromatography (SEC) to probe the conformation and oligomeric state of SpoT_{Ab} in solution (FIG. 1e-f).

The SAXS data revealed that in solution SpoT_{Ab} has an oblate shape compatible with the structure determined by X-ray. Both SAXS and SEC consistently support the monomeric nature of SpoT_{Ab}, even at concentrations as high as 8 mg/mL. Both the molecular weight of ˜90 kDa by SEC as well the estimates of Mw of ˜85 kDa and Rg of 34.9 Å by SAXS (FIG. 1e-f) agree with the 80 kDa theoretical molecular weight of monomeric SpoT_{Ab}. Furthermore, the analysis of the normalised Kratky plot derived from the scattering curve lends further support for a compact monomeric structure of SpoT_{Ab} in solution (FIG. 1f), and the ab initio envelope calculated from the experimental SAXS data (FIG. 1g) is compatible with the τ-shaped structure of SpoT_{Ab} determined by X-ray. Collectively these results demonstrate that in solution the monomeric SpoT_{Ab} adopts a conformation that is very similar to the i-shaped conformation observed in the crystal with the HD domain protruding from the disc-shaped enzyme.

### 5. The Enzymatically-Inactive Pseudo-SYNTH of SpoT_{Ab} is a Regulatory Domain

In the monofunctional stringent factor RelA, the enzymatically inactive pseudo-HD domain has evolved into a regulatory domain controlling catalysis via an intra-NTD allosteric regulatory mechanism (Roghanian et al., Mol Cell, 2021; Sinha and Winther, Commun Biol, 2021). This is also the case with the specialisation of SpoT_{Ab} as a monofunctional hydrolase where the pseudo-SYNTH domain has evolved into a strictly regulatory/structural domain. Superposition of the SYNTH domain from Rel_{Tt} onto the pseudo-SYNTH domain of SpoT_{Ab} reveals extensive reorganisation of the vestigial catalytic domain in SpoT_{Ab}, consistent with differential conservation patterns in the G-loop and the ATP recognition motif. These involve the residues that coordinate adenosine and guanosine (R249 to N241, R277 to E267 and Y329 to N304) and the majority of phosphate-coordinating groups. Crucially, the catalytic residues D272 and Q347 are substituted for S263 and T321, respectively. These substitutions essentially impede the deprotonation and activation of the 3′-OH of GD(T)P, and Mg²⁺ binding, precluding the nucleophilic attack on the β-phosphate of ATP. We directly probed GDP binding by SpoT_{Ab}^{NTD} and RelA_{Ab}^{NTD} by ITC. As expected, while SpoT_{Ab} does not bind GDP, RelA_{Ab} binds GDP with an affinity of 62 μM, which is similar to our earlier estimates for RelA_{Ec}^{NTD} and Rel_{Bs}^{NTD} (Takada et al., Nucleic Acids Res, 2021; Roghanian et al., Mol Cell, 2021).

### 6. SpoT_{Ab} is not Allosterically Regulated by the Alarmone pppGpp

The enzymatic activity of long RSHs is regulated via strong allosteric coupling between the HD and SYNTH domains that results in antagonistic conformational states (Hogg et al., Cell, 2004; Tamman et al., Nat Chem Biol, 2020; Roghanian et al., Mol Cell, 2021). While in Rel/RelA (p)ppGpp bind the hinge region connecting the SYNTH and HD/pseudo-HD domains to stimulate the SYNTH activity, this regulation is lost in SpoT_{Ec} (Roghanian et al., Mol Cell, 2021). Our structure of SpoT_{Ab} provides a mechanistic interpretation. In the τ-state the highly structured Core subdomain makes numerous contacts with SYNTH providing further scaffolding to the already more stable version of the HD:SYNTH hinge of SpoT_{Ab}. Additionally, there are several important substitutions in the (p)ppGpp binding site that would be expected to compromise (p)ppGpp binding and alarmone-mediated regulation, specifically in Q203 (a residue involved in ribose coordination and strictly conserved as A in RelA (Roghanian et al., Mol Cell, 2021)) and in T209 (a residue involved in phosphate coordination, typically K or R in RelA (Roghanian et al., Mol Cell, 2021)).

To directly validate the lack of pppGpp-mediated regulation in SpoT_{Ab}, we characterised the interaction between pppGpp and SpoT_{Ab}^{NTD} by ITC. As expected, SpoT_{Ab}^{NTD} does not bind pppGpp allosterically. Following the experimental approach used earlier for SpoT_{Ec} (Roghanian et al., Mol Cell, 2021), we next grafted the allosteric site of A. baumannii RelA (²³⁶RelA²⁴⁶) onto SpoT_{Ab}^{NTD} (replacing ²⁰¹SpoT_{Ab}²¹¹). Just as in the case of SpoT_{Ec}, this resulted in a RelA-like affinity to pppGpp of the chimera RSH (KD=5.6 μM). Collectively, these results support the generality of alarmone-mediated control being lost in SpoT and only present in SYNTH-active Rel/RelA stringent factors that mediate acute stringent response upon amino acid starvation.

### 7. The Dipolar Architecture of the HD Active Site is Conserved Between Rel and SpoT

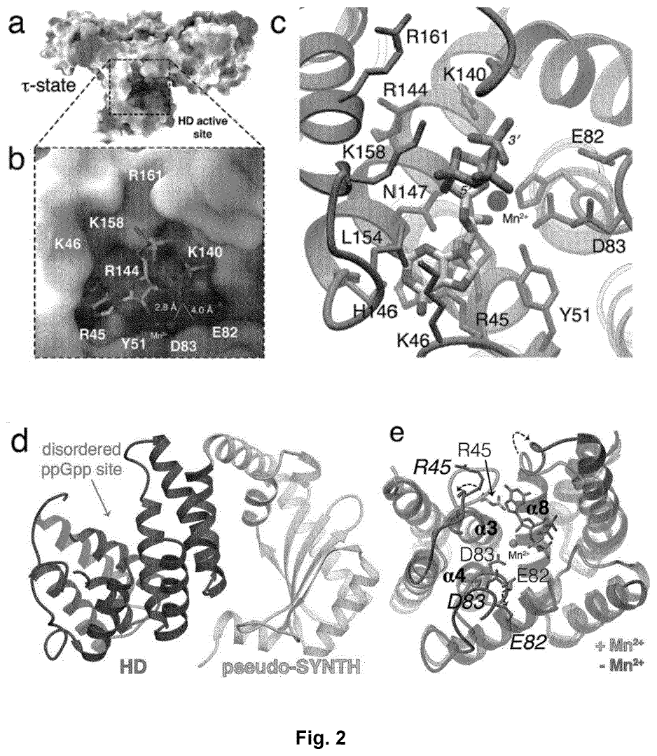

Inspection of the electron density map of the SpoT_{Ab}-ppGpp complex reveals that the alarmone is bound in high occupancy in each of the four SpoT_{Ab} molecules present in the asymmetric unit of the crystal, with the coordination of the guanine base of ppGpp (FIG. 2a-c) resembling that observed in Rel_{Tt}^{NTD}-ppGpp 23 and Rel_{Tt}^{NTD}-pppGpp complexes (Mojr et al., ACS Chem Biol, 2021). We probed enzymatically the role of each residue involved in guanine coordination via systematic Ala-substitutions. While substitution of R45 (stacking the guanine) abrogated hydrolysis, removing Van der Waals contacts to L154 decreased the activity approximately two-fold; interactions with K46 were redundant. Disruption of the hydrogen bond of the guanine to T150 had only a minor effect. The additional hydrogen bond formed between the carbonyl group of the guanine and the enzyme's backbone likely accounts for the guanine specificity of SpoT over adenosine.

As observed earlier for Rel_{Tt}^{NTD} (Tamman et al., Nat Chem Biol, 2020), the hydrolase active site of SpoT_{Ab} displays a dipolar charge distribution with a highly basic half mediating the stabilization of the 5′- and 3′-polyphosphate groups of the substrate and the other highly acidic half mediating the 3′-pyrophosphate hydrolysis (FIG. 2a-b). Closer inspection of the complex reveals the crucial role of Y51 and the 82ED83 active site motifs as they work together with the Mn²⁺ cofactor to coordinate and stabilise a network of water molecules near the sugar-phosphate moiety during hydrolysis (FIG. 2b-c). Indeed, substitutions of Y51, E82, D83 or N147 render SpoT_{Ab} HD inactive in our enzymatic assays. At the positively charged side active site the 5′-polyphosphate is loosely coordinated and exposed to the bulk solvent. By contrast K140 and R144 hold the 3′-pyrophosphate in place during hydrolysis and Ala substitutions of these residues decrease the activity of the enzyme between 5- and 10-fold suggesting these are key residues that orient the scissile bond.

### 8. Mn²⁺ Ion Organizes the HD Active Site of SpoT_{Ab}

The essential role of the divalent manganese ion Mn²⁺ in (p)ppGpp pyrophosphate hydrolysis is well documented for both Rel (Hogg et al., Cell, 2004; Takada et al., Nucleic Acids Res, 2021; Avarbock et al., Biochemistry, 2000; Van Nerom et al., Acta Crystallogr F Struct Biol Commun, 2019) and SpoT_{Ec} (Heinemeyer et al., Eur J Biochem, 1978). Our isothermal titration calorimetry (ITC) measurements demonstrate that unliganded, metal-free SpoT_{Ab}^{NTD} binds Mn²⁺ with a KD of 35.3 M. Furthermore, while metal-free full-length SpoT_{Ab} is completely HD inactive, the HD activity is readily restored upon addition of Mn²⁺.

To directly reveal the structural role of Mn²⁺ we determined the X-ray structure of SpoT_{Ab}^{NTD} in the metal-free state (FIG. 2d). Comparison with the structure of the SpoT_{Ab}-ppGpp complex provides a structural explanation for the essentiality of Mn²⁺ for catalysis: in addition to its role in hydrolysis, by connecting α3, α4 and α8, Mn²⁺ coordination brings together the two halves of the HD domain and provides structural support to the active site (FIG. 2d-e). While the overall topology of the SpoT_{Ab} HD domain is similar to that of Mn²⁺-liganded Rel_{Tt}^{NTD} 23, the removal of the metal ion has a profound effect on the local conformation of the active site of SpoT_{Ab}^{NTD}. The catalytic 78HD79 and 82ED83 motifs are largely misaligned, loops S110-Y117 and A153-K158 that are involved in the 3′- and 5′-phosphate coordination are disordered, and the guanine-coordinating loop T44-Y51 assumes a conformation incompatible with the base coordination (FIG. 2e). Importantly, all of these changes do not result in the opening of the enzyme's^{NTD} that was observed in Rel^{Tt} upon removal of Mn²⁺ (Tamman et al., Nat Chem Biol, 2020). These observations suggest that with the evolution as a monofunctional enzyme, SpoT_{Ab} shed the allosteric conformational control between the HD and pseudo-SYNTH domains.

### 9. The CTD Allosterically Stimulates the Hydrolysis Activity of the SpoT NTD

Until now, our understanding of the function of the CTD region of long RSHs was based exclusively on studies of Rel and RelA. This has established a role of the CTD in the association of the stringent factors with starved ribosomes resulting in the activation of the SYNTH activity and the auto-inhibition of the factor's SYNTH activity off the ribosome (Arenz et al., Nucleic Acids Res, 2019; Loveland et al., Elife, 2016; Pausch et al., Cell Rep, 2020; Mechold et al., J Bacteriol, 2002; Takada et al., Nucleic Acids Res, 2021). Weak hydrolase activity of the CTD-truncated Rel has also indicated a possible HD-stimulatory role of the CTD through an intra-molecular regulation of the hydrolase function (Takada et al., Nucleic Acids Res, 2021; Ronneau et al., Nucleic Acids Res, 2019; Takada et al., Front Microbiol, 2020), suggesting that a similar mechanism could also be at play in the case of SpoT.

To probe this hypothesis, we characterised the HD activity—both in vitro and in vivo—of a set of progressively C-terminally truncated variants of SpoT_{Ab} lacking i) RRM (SpoT_{Ab}¹⁻⁶¹⁴, amino acids 1-614), ii) RRM and ZFD (SpoT_{Ab}¹⁻⁵⁶⁰), iii) RRM, ZFD and HEL (SpoT_{Ab}¹⁻⁴⁵⁴), iv) CTD altogether, i.e. RRM, ZFD, HEL and TGS (SpoT_{Ab}¹⁻³⁸⁵), v) CTD as well as the Core domain (SpoT_{Ab}¹⁻³³⁹) and finally, a variant that consisted of just the HD domain (SpoT_{Ab}¹⁻¹⁹⁵). These truncated variants were all generated at the endogenous SpoT locus in a ΔrelA Ptac::relA A. baumannii strain, and the ability to grow on complex media supplemented with IPTG was evaluated as a proxy of the (p)ppGpp hydrolase activity of SpoT_{Ab} in vivo.

While SpoT_{Ab} variants lacking the RRM or the RRM and ZFD domains retained wild-type ability to sustain the bacterial growth grow—i.e. could efficiently degrade (p)ppGpp synthesised by RelA—further C-terminal truncations compromised the in vivo HD functionality, as evidenced from pronounced growth defects. Biochemical assays are in agreement with the in vivo data. Truncation of the RRM and ZFD decreases the HD activity 5-fold. Further deletion of the TGS-HEL domains leads to a dramatic 42-fold decrease in activity. Truncations beyond the TGS compromised the activity by 70-fold or more and isolated HD domain was nearly inactive. Collectively, our results suggest that the CTD region functions as an allosteric activator of the hydrolase function of SpoT_{Ab}. Next, we set out to dissect the molecular mechanism of the CTD-mediated NTD control and assign the molecular functions to individual CTD domains.

### 10. The Core Domain is a Linchpin that Controls the τ-State

Both the overall structural arrangement of SpoT_{Ab} and our sequential domain truncation experiments suggest that the Core-mediated allosteric crosstalk between the HD and rest of the domains of the enzyme is essential for enzyme's functionality. To specifically assess the role of the individual interdomain interactions we introduced single point substitutions at each of the interfaces of the Core with regulatory CTD domains and measured the hydrolase activity of the SpoT_{Ab} variants. An intact HD:Core:TGS interface—the structure involved in scaffolding the HD active site—is crucial for HD activity, as the Y375G substitution at the HD:Core:TGS resulted in a 5-fold decrease in activity compared to the wild type. While substitutions at the ZFD (L373G/D374G) and RRM (A351K) domain interfaces also resulted in a pronounced defect (19 and 3-fold decrease, respectively), perturbations at the Core:pseudo-SYNTH domain interface (A348R) had only a minor effect on hydrolysis. Finally, decoupling the contacts of HD from the τ-cap via the L356D substitution, located at the interface between Core domain and α6-α7 motif of HD (Tamman et al., Nat Chem Biol, 2020), has a dramatic 35-fold decrease in HD activity, suggestive of an allosteric signal transduction path between the cap and stem regions of the enzyme. When we monitored the thermodynamic stability of these Core variants of SpoT_{Ab} we observed they all have lower stability and loss of structure compared to the wild type. This suggests that an increase in the configurational entropy of the Core has a global effect in the dynamics and compactness of the enzyme. The existence of an allosteric relay mediating a CTD-dependent activation of HD via the Core is further supported by the consistent decrease in hydrolysis associated with the aforementioned C-terminal truncations that affect the feedback of the Core to the HD, as well as by the observation that the deletion of domains HEL and TGS results in a 50-fold decrease in activity despite the presence of the other regulatory domains (pseudo-SYNTH, ZFD and RRM).

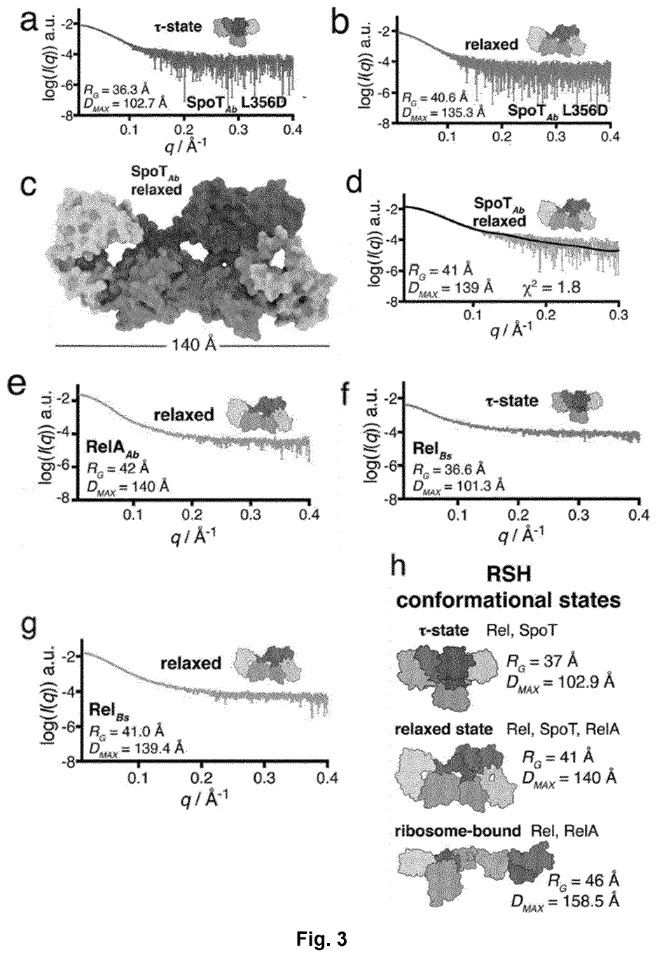

We next used SEC-SAXS to directly probe the role of each contact at the interface of the Core with the different domains of SpoT_{Ab} on stabilisation of the τ-state. The L356D substitution (SpoT_{Ab}^{L356D}) results in the segregation of the population into two conformational states with major differences in RG (radius of gyration) and particle dimensions (DMAX). In SpoT_{Ab}^{L356D} one state is the compact τ-shape observed in the crystal structure (FIG. 3a), while the other state is more relaxed (RG=41 Å, DMAX=130 Å) with dimensions reminiscent of that of the less compacted Rel and RelA—but not quite as elongated as in the ribosome-bound state (FIG. 3b). In this relaxed state the Core and HEL domains appear to have transitioned to a more disordered state that is consistent with the conformational states of these regions in the fully elongated state observed in Rel/RelA (FIG. 3c-d); the other domains retain their structural integrity. Prompted by this analogy, we next probed A. baumannii monofunctional synthetase RelA and B. subtilis bifunctional RSH Rel_{B}s with SAXS. The dimensions of RelA_{Ab} (RG=42 Å, DMAX=130 Å, Mw=88 kDa) are consistent with that of the relaxed state of SpoT_{Ab}^{L356D}, whereas Rel_{Bs} is populated by both the relaxed and τ-states (FIG. 3e-g).

Collectively, our results suggest that the Core domain functions as an allosteric relay that conveys signals from the CTD to the HD. At the structural level the composition of the Core is the key to the conformational state of the enzyme as defined by the three major conformations observed in SpoT, Rel and RelA (FIG. 3h). The correlation of the decrease in HD activity with entropy-increasing substitutions such as A351K, L356D, L371G/D374G, and Y375G supports the notion that the increase in structural disorder or flexibility of the Core domain (or the other IDRs) likely drives the conformational equilibrium of the enzyme away from the τ-state. This decrease in activity observed by the disruption of the τ-shape is also consistent with the lack of hydrolysis in Rel homologues that underwent an order-to-disordered transition while accommodating in ribosomal A site (Takada et al., Nucleic Acids Res, 2021). In this context the aforementioned relaxed state is likely the idle resting state of long RSH enzymes in which the CTD precludes the function of SYNTH while not activating HD.

### 11. The TGS Domain Acts as a Scaffold for the HD Active Site

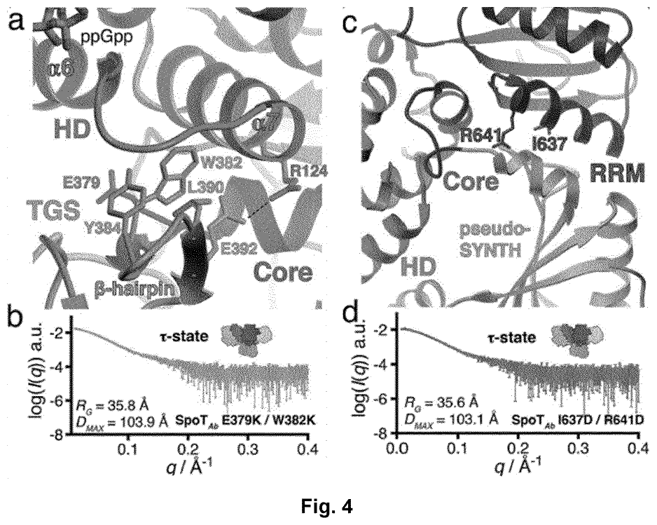

The α6-α7 element plays a crucial role in the allosteric regulation of the opposing activities of bifunctional Rel_{Tt} (Tamman et al., Nat Chem Biol, 2020). In Rel_{Tt}, α6-α7 of projects away from the HD catalytic centre to accommodate the 3′ and 5′ polyphosphate groups as well as allowing the catalytic ⁸²ED⁸³ motif to get in position, close to the 3′ phosphates, priming the enzyme for hydrolysis. In SpoT_{Ab} the outward-pointing conformation of α6-α7 is further stabilised by the N-terminal region of the TGS and the Core domains which function as a clamp to keep α6-α7 in the HD-compatible position, with the HEL domain providing an additional support via the Core (FIG. 4a). The dramatic drop in the activity of the SpoT_{Ab} variant lacking the TGS and HEL domains (FIG. 4d) substantiates the functional importance of this stabilising effect.

At the HD:TGS interface the β-hairpin of the TGS—the very element which is involved in tRNA recognition in Rel (Pausch et al., Cell Rep, 2020; Takada et al., Nucleic Acids Res, 2021; Takada et al., Front Microbiol, 2020) and RelA (Brown et al., Nature, 2016; Loveland et al., Elife, 2016; Winther et al., Mol Cell, 2018)—is buried and stacking directly the α6-α7 element via a small hydrophobic interface formed by W382, Y384, L390 and the R124-E392 salt bridge (FIG. 4a). Disrupting this interface with the E379K/W382K substitutions (SpoT_{Ab}^{E379K/W382K}) led to a 17-fold decrease in the hydrolase activity of the enzyme suggesting that the HD:TGS interface constitutes as an important allosteric signal transduction pathway. This scaffolding role is complemented by the Core that wraps tightly around α7 thus preventing the recoil of α6-α7 away of the HD active site, which, as we observed earlier in Rel_{Tt} (Tamman et al., Nat Chem Biol, 2020), induces the opening of the NTD. Indeed, substitutions at the Core:α6-α7 interface such as the aforementioned Y375G also affected hydrolysis. Interestingly, despite the strongly attenuated HD activity of SpoT_{Ab}^{E379K/W382}K, SAXS showed SpoT_{Ab}^{E379K/W382K} remains in the τ-state (RG=35 Å, DMAX=104 Å), suggesting an allosteric communication via the HD:Core:TGS axis (FIG. 4b).

Given that SpoT_{Ab} is SYNTH-inactive and is not specifically regulated by tRNA or ribosomes (FIG. 1d), it is not surprising that TGS residues involved in tRNA recognition—such as the crucial His residue involved in the recognition of the 3′ CCA end by Rel (Pausch et al., Cell Rep, 2020; Takada et al., Nucleic Acids Res, 2021; Takada et al., Front Microbiol, 2020) and RelA (Brown et al., Nature, 2016; Winther et al., Mol Cell, 2018) (S407 in SpoT_{Ab})—are lost in the monofunctional SpoT_{Ab} (but are present in bifunctional SpoT_{E}C (Atkinson et al., PLoS One, 2011)). Moreover, the τ-state is sterically incompatible with the potential recognition of tRNA by TGS due to sequestration the β-hairpin and α-helical elements. All these observations suggest that in SpoT_{Ab} the TGS has been repurposed as a scaffolding domain crucial to sustain hydrolysis, with both TGS and Core cooperating to lock the α6-α7 in place, stabilising the HD active site. This contrasts with its crucial function of recognition of uncharged tRNA in Rel/RelA (Brown et al., Nature 2016; Loveland et al., Elife, 2016; Pausch et al., Cell Rep, 2020; Takada et al., Nucleic Acids Res, 2021, Winther et al., Mol Cell, 2018).

### 12. The ZFD and RRM Domains Finetune the Hydrolytic Activity of SpoT_{Ab}

With ZFD and RRM positioned close to the disc-shaped cap and connecting with the pseudo-SYNTH domain, the resulting inter-domain interfaces are likely to play a role in the stability the τ-state as well as to allosterically control of HD via the HD:pseudo-SYNTH relay. In agreement with this hypothesis, disruptive substitutions at the Core:HD (L356D), Core:pseudo-SYNTH:RRM (A351K) and Core:ZFD (L373G/D374G) that decreased the stability of the τ-state also decreased the HD activity of the enzyme by 35-, 3- and 22-fold, respectively. Therefore, we reasoned that substitutions stabilising the Core:pseudo-SYNTH:RRM and Core:ZFD interfaces would, conversely, trigger an allosteric activation of hydrolysis.

To probe this hypothesis, we introduced substitutions that would increase the contacts of RRM with pseudo-SYNTH via hydrogen bonds, I637D/R641D, and the Core with the ZDF, D374R (FIG. 4c). Denaturation experiments showed SpoT_{Ab}^{D374R} and SpoT_{Ab}^{I637D/R641D} have higher stability and compactness than the WT and SAXS measurements on SpoT_{Ab}^{I637D/R641D} confirmed this variant retained the τ-state (FIG. 4d). As expected, the HD turnover of both enzyme variants increased (by 2.1- and 1.6-fold, respectively), and both behave like wild-type SpoT_{Ab} in vivo.

Collectively, our results establish that HD activity is coupled to the stability of the τ-state, with the Core domain working as an allosteric transducer that allows the catalytic HD to communicate with all the regulatory domains. Substitutions or interactions that stabilise the τ-state increase hydrolysis, whereas τ-state-destabilising substitutions lower the HD activity.

### 13. An Intact τ-Shaped SpoT_{Ab} is Required for Virulence of A. baumannii

Functional (p)ppGpp-mediated signalling plays a crucial role in antibiotic tolerance and virulence of A. baumannii (Perez-Varela et al., J Bacteriol, 2020; Kim et al., Virulence, 2021). We used the wax moth G. mellonella larvae infection model to assess the functionality of mutant spoT_{Ab} variants in supporting virulence of A. baumannii AB5075. Only the strain with wild type-like virulence was the one expressing SpoT_{Ab} D374R variant with a HD activity slightly higher than that of the WT SpoT. The spoT_{Ab}^{D374R} strain has rapidly killed 100% of the larvae within the first two days whereas 60% of the larvae survived 6 days of infection with the (p)ppGpp®⁰ ΔrelA strain. Infection with A. baumannii expressing the ΔRRM-truncated enzyme SpoT_{Ab}¹⁻⁶¹⁴ resulted in 25% survival rate of larvae after 6 days. Notably, the RRM-truncated SpoT_{Ab}¹⁻⁶¹⁴ had 6-fold lower hydrolase activity as compared to wild type, and the strain displays no growth defects when grown on LB plates. The defect in virulence becomes more prominent with truncations beyond the TGS domain: SpoT_{Ab}¹⁻⁴⁵⁴ and SpoT_{Ab}¹⁻³³⁹. The strong decrease in HD activity associated with the A. baumannii strains expressing these SpoT variants results in 100% survival of the infected larvae. Collectively our results suggest that while a basal level of the HD hydrolase activity is sufficient to sustain bacterial growth in non-stressed conditions (e.g. on a plate and in liquid culture), the pathogen requires fully functional -TD- and Core-mediated control of SpoT_{Ab} to tune the HD activity and efficiently establish a successful infection.

### 14. Discussion of Examples 1-13

This study reveals the unexpected τ-shaped architecture of full-length monofunctional SpoT_{Ab}, which enables auto-stimulation of the hydrolase activity of the enzyme by its CTD. With the loss of the synthetase function, the pseudo-SYNTH domain of SpoT_{Ab} becomes a regulatory and structure stabilising domain. Together with TGS, HEL, ZFD and RRM, pseudo-SYNTH defines the interaction network that transmits the allosteric signal from the CTD to the HD active site via the Core of the enzyme, to regulate (p)ppGpp hydrolysis. The Core element, together with the TGS and Mn²⁺, aligns the active site residues of the HD in the correct position for catalysis. Compromising the functionality of either of these elements through substitutions of key residues results in major defects in hydrolysis activity. By contrast, pseudo-SYNTH, ZFD and RRM all subtly tune the HD activity of SpoT_{Ab} up or down by modulating its interactions with the Core. Interestingly, the ribosome-associated Rel/RelA (p)ppGpp synthetases, lacking the Core are non-functional in vivo and SYNTH-inactive, with the minimal enzyme version with SYNTH activity consisting of HD/pseudo-HD, SYNTH and Core domains (Hogg et al., Cell, 2004; Takada et al., Nucleic Acids Res, 2021; Roghanian et al., Mol Cell, 2021; Ronneau et al., Nucleic Acids Rest, 2019). Therefore, the presence of the Core and its crosstalk with the HD/pseudo-HD domain likely constitutes universal structural requirement for the efficient stabilization of the active states of long RSH enzymes.

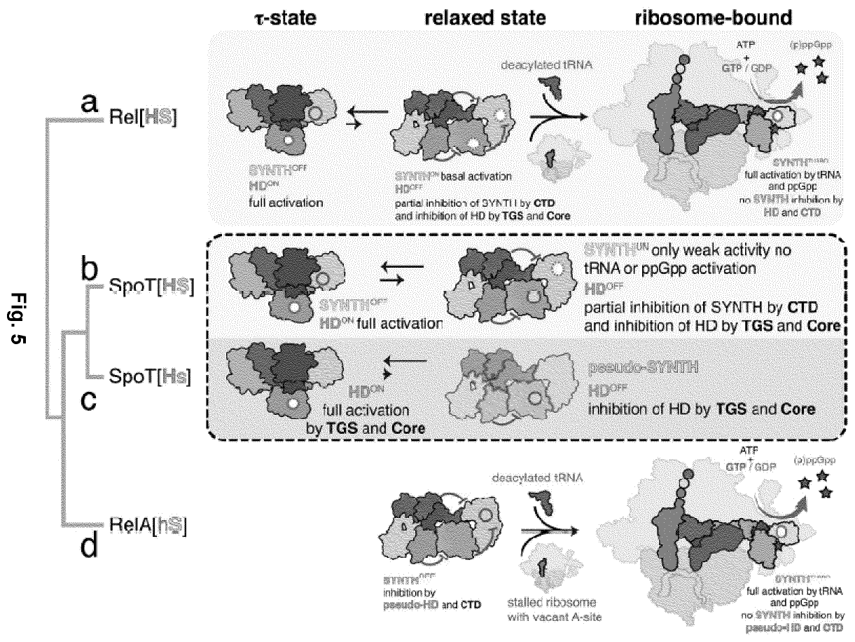

We propose a unifying scheme that rationalises the evolution of the enzymatic output of long RSHs through fine-tuning of the conformational equilibrium of the τ, relaxed and ribosome-bound states of these enzyme (FIG. 5). The very presence of catalytically-competent synthetase and hydrolase domains in bifunctional Rel[HS] and SpoT[HS] requires both the τ and relaxed states as part of the conformational spectrum of these enzymes (FIG. 5a-b). While the τ-state primes Rel/SpoT for efficient (p)ppGpp hydrolysis, the more elongated relaxed state sets the enzyme for low-efficiency (p)ppGpp synthesis. To fully activate its SYNTH activity, the enzyme needs to be further stimulated by starved ribosomes to attain the highly elongated ribosome-bound state; this transition is possible for amino acid starvation sensor Rel[HS], but not for SpoT, which is not under allosteric control by starved ribosomes and ppGpp (Roghanian et al., Mol Cell, 2021) (FIG. 5a). In the further subfunctionalised enzymes (dedicated hydrolase Moraxellaceae SpoT[Hs] and dedicated synthetase RelA[hS]) the intrinsic structural equilibrium is limited to a subset of the conformations accessible to ancestral bifunctional Rel[HS](FIG. 5c-d). Compared to SpoT[HS], in SpoT[Hs] the equilibrium is further shifted towards the HD-active τ-state required for hydrolysis (FIG. 5c). In contrast, in RelA[hS] the τ-state becomes inaccessible, and the enzyme is primed for ribosomal recruitment upon which it is stabilised in the highly elongated ribosome-bound SYNTH-active state (FIG. 5d).

Expansion/contraction of the disordered regions is the likely molecular driver of the fine-tuning of the enzymatic output in long RSHs through the restriction of the conformational space. Longer IDRs favour the relaxed state in RelA[hS] and increase the frustration of the enzyme, whereas the shorter IDRs favour the compact HD-active τ-state in SpoT[Hs]. This genetic finetuning of a catalytic function, based on the optimization of the length and the forces generated by intrinsically disordered regions, is reminiscent of the evolution of human glucocorticoid receptor isoforms (Li et al., Elife, 2017) or the UDP-α-d-glucose-6-dehydrogenase (Keul et al., Nature, 2018). Such mechanisms seem to have evolved as a solution for conformationally heterogenous proteins with partially active resting states, that are under strong energetic and functional frustration.

The unifying scheme presented here provides a framework that can be used to rationalise the “hub” nature of SpoT and how binding partners such as the Acyl Carrier Protein (ACP) and the Regulator of RpoD-σ⁷⁰-(Rsd) could modulate its output (Battesti and Bouveret, Mol Microbiol, 2006; Lee et al., Proc Natl Acad Sci USA, 2018) or in the case of Rel/RelA how the ribosome prevents hydrolysis by exploiting this extensive allosteric network. Other protein partners of Rel such EIIA^{NTR} and DarB (Ronneau et al., Nucleic Acids Res, 2019; Kruger et al., Nat Commun, 2021) could also modulate the intramolecular allosteric communication of the regulatory domains with HD by favouring of the i- or relaxed states, thus conditioning the catalytic output of the enzyme.

### 15. Key Amino Residues for Candidate Compound Screening

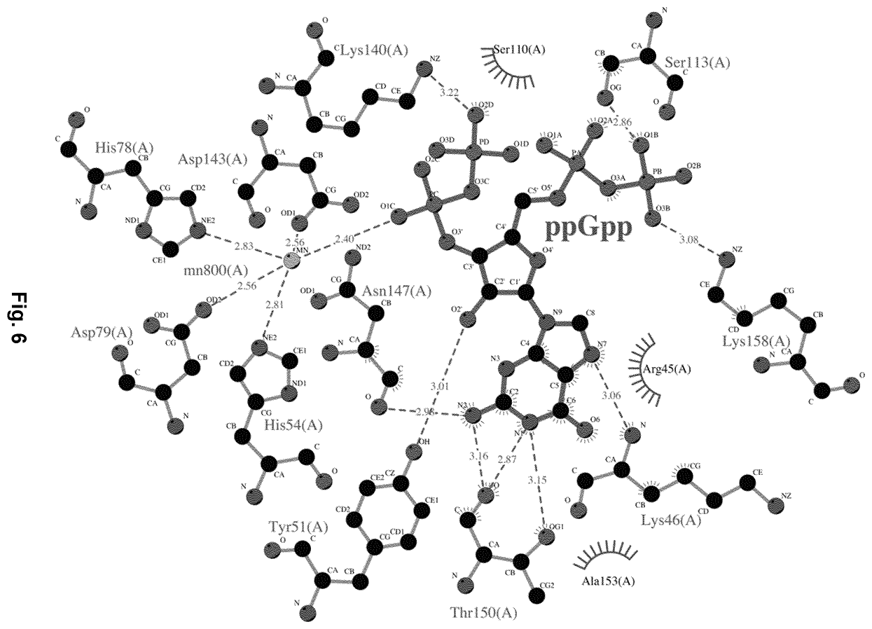

Based on the above findings, the inventors are able to identify a selection of key residues which are preferred residues for a successful candidate compound (i.e. a candidate A. baumannii SpoT enzyme modulator) to bind with (FIG. 6).

Lengthy table referenced here
US20260128119A1-20260507-T00001
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260128119A1). An electronic copy of the table will also be available from the USPTO upon request and payment of the fee set forth in 37 CFR 1.19(b)(3).

1. A method for identifying compounds that modulate A. baumannii SpoT activity comprising the step of employing a three dimensional structure represented by a set of atomic coordinates presented in Table 1 or a subset thereof, or atomic coordinates which deviate from those in Table 1 or a subset thereof by a root mean square deviation (RMSD) of residue over protein backbone atoms by no more than 3 Å and assessing the degree of fit of a candidate compound to said three-dimensional protein structure of A. baumannii SpoT.

2. The method according to claim 1, wherein the method is a method for identifying compounds that modulate A. baumannii SpoT hydrolase activity.

3. The method according to claim 1, wherein interactions of said candidate compound with one or more amino acid residues of a region on the surface of the protein defined by amino acid residues: Arg45, Lys46, Ser47, Tyr51, His54, His78, Asp79, Ser113, Lys140, Asp143, Asn147, Thr150, Ala153, or Lys158 of the SpoT amino acid sequence as defined by an amino acid sequence with at least 70% sequence identity to the amino acid sequence of SEQ ID NO: 1 indicate the candidate compound is a modulator of SpoT hydrolase activity.

4. The method according to claim 1, wherein the amino acid sequence has at least 80% sequence identity to the amino acid sequence of SEQ ID NO: 1.

5. The method according to claim 1, wherein the amino acid sequence comprises, consists essentially of, or consists of the amino acid sequence of SEQ ID NO: 1.

6. The method according to claim 1, further comprising determining a score of said candidate compound to modulate A. baumannii SpoT activity based on the number of interactions with said amino acid residues.

7. The method according to claim 1, further comprising comparing the conformational state of A. baumannii SpoT before and after said candidate compound binds to A. baumannii SpoT, wherein a change in conformational state is indicative for the candidate compound to be a bona fide modulator of A. baumannii SpoT activity.

8. The method according to claim 1, wherein the method is a method for identifying compounds that decrease A. baumannii SpoT hydrolase activity.

9. The method according to claim 1, which is a computer-implemented method, said computer comprising an inputting device, a processor, a user interface, and an outputting device, wherein said method comprises the steps of:
a) generating a three-dimensional structure of the atomic coordinates of Table 1, or a subset thereof;
b) fitting the structure of step a) with the structure of a candidate compound by computational modeling; and
c) selecting a candidate compound that possesses energetically favorable interactions with the structure of step a).

10. The method according to claim 9, wherein said fitting comprises superimposing the structure of step a) with the structure of said candidate compound.

11. The method according to claim 9, wherein said candidate compound of step c) can bind to at least 1 amino acid residue of the structure of step a) without steric interference.

12. (canceled)

13. A crystal of A. baumannii SpoT-ppGpp complex, comprising a structure characterized by the atomic coordinates or a subset thereof as defined in Table 1.

14. An in vitro method for identifying a compound which specifically modulates A. baumannii SpoT hydrolase activity, comprising the steps of:
a) providing a candidate compound;
b) providing an A. baumannii SpoT polypeptide or SpoT-ppGpp complex;
c) contacting said candidate compound with said A. baumannii SpoT polypeptide or SpoT-ppGpp complex;
d) determining the hydrolase activity of A. baumannii SpoT in the presence and absence of said candidate compound; and
e) identifying said candidate compound as a compound which modulates A. baumannii SpoT if a change in hydrolase activity is detected.

15. A computer system, intended to generate three dimensional structural representations of an A. baumannii SpoT protein and/or SpoT-ppGpp complex, complexes of A. pbaumannii SpoT protein with binding compounds or modulators for analyzing or optimizing binding of compounds or modulators to said A. baumannii SpoT protein and/or SpoT-ppGpp complex, the system containing computer-readable data comprising one or more of:
(a) the coordinates of the A. baumannii SpoT protein structure listed in Table 1, optionally varied by a root mean square deviation of residue backbone atoms of not more than 3 Å, or selected coordinates thereof,
(b) the coordinates of the A. baumannii SpoT-ppGpp complex structure listed in Table 1, optionally varied by a root mean square deviation of residue backbone atoms of not more than 3 Å, or selected coordinates thereof;
(c) the coordinates of a candidate binding compound or modulator generated by interpreting X-ray crystallographic data, cryo-EM or NMR data by reference to the coordinates of the A. baumannii SpoT protein structure and/or SpoT-ppGpp complex structure, listed in Table 1, optionally varied by a root mean square deviation of residue backbone atoms of not more than 3 Å, or selected coordinates thereof, and
(d) structure factor data derivable from the coordinates of (a), (b) or (c).

16. The method according to claim 1, wherein the amino acid sequence has at least 85%-sequence identity to the amino acid sequence of SEQ ID NO: 1.

17. The method according to claim 1, wherein the amino acid sequence has at least 90% sequence identity to the amino acid sequence of SEQ ID NO: 1.

18. The method according to claim 1, wherein the amino acid sequence has at least 95%-sequence identity to the amino acid sequence of SEQ ID NO: 1.

19. The method according to claim 1, further comprising determining a score of said candidate compound to modulate A. baumannii SpoT hydrolase activity based on the number of interactions with said amino acid residues.

20. The method according to claim 7, wherein the conformational state of A. baumannii SpoT before candidate compound binding is the conformational state characterized by the atomic coordinates of Table 1.

21. The method according to claim 10, wherein said fitting comprises superimposing the structure of the atomic coordinates corresponding to bound ppGpp with the structure of said candidate compound.


SEQUENCE LISTING (1 sequence)

SEQ ID NO: 1 - protein, 701 aa, Acinetobacter baumannii
  source 1..701: note = SpoT enzyme
MPGEEVSQAKQQLKLIIDPYLSVSEVEKVLAACDFGDLAHTGITRKSGEPYILHPIAVSCILANMRLDPETLMAALLHDVIEDTQYTKDDIIERFGQTVAELVDGVTKLSQSSDKEYNKAASFRKILQATLQDPRVIIIKLADRYHNMTTLGALRPDKRARIAQETFDIFVPMARLVGMNEMADNLENLCYQNLDLDMFDNVQNALLQTKPERCKYQSIWEQNLAELLHNYHIQGRIKKKNNNIELLRHFVKNEMDLQELTHSHAFEIVLQSIADCDRLVAALKENFQVIQYQDHIRRPLPGGNQSLMIKLKGEKTTLSLTIQTELMRKAARFGVVLGENAPQTCRSAIQASMQNLNTLIDGECAKTTFNDLLDYLHQEKIWVYTPHGQLHELPQGATVVDFAYSASLFLGNHAVGAKVDGEIKPLSTPLVSGQVIEIITDVLATPNPDWLSFINTQKARRALQHVLKDQDIEEQRLVGAQALSRALKLFNRSINDLSDADWLDLLQWRHIDNKDALFEQIAVGDLLPQLVANHLFANDKHPRAENSDRLIQGTEGIDVKYAHCCNPILGDPIQGHLTRRGLIVHRIRCHNLLHEQHLHPENIMPLQWKADDVDDVRFTAYLAIYMAMNDEQVSDLIYQCRKNNAGVEMVHSNEQRTFVNIVVNNRKHIAKVIRDLRMHYGFPRIERLDAPAPQMEISKVS